SEQUENCE LISTING (21 sequences)

SEQ ID NO: 1 - DNA, 1590 bp, Homo sapiens
gccagatctgtgagcccagcgctgactgcgccgcggagaaagccagtgggaacccagacccataggagacccgcgtccccgctcggcctggccaggccccgcgctatggagttcctctgggcccctctcttgggtctgtgctgcagtctggccgctgctgatcgccacaccgtcttctggaacagttcaaatcccaagttccggaatgaggactacaccatacatgtgcagctgaatgactacgtggacatcatctgtccgcactatgaagatcactctgtggcagacgctgccatggagcagtacatactgtacctggtggagcatgaggagtaccagctgtgccagccccagtccaaggaccaagtccgctggcagtgcaaccggcccagtgccaagcatggcccggagaagctgtctgagaagttccagcgcttcacacctttcaccctgggcaaggagttcaaagaaggacacagctactactacatctccaaacccatccaccagcatgaagaccgctgcttgaggttgaaggtgactgtcagtggcaaaatcactcacagtcctcaggcccatgacaatccacaggagaagagacttgcagcagatgacccagaggtgcgggttctacatagcatcggtcacagtgctgccccacgcctcttcccacttgcctggactgtgctgctccttccacttctgctgctgcaaaccccgtgaaggtgtatgccacacctggccttaaagagggacaggctgaagagagggacaggcactccaaacctgtcttggggccactttcagagcccccagccctgggaaccactcccaccacaggcataagctatcacctagcagcctcaaaacgggtcagtattaaggttttcaaccggaaggaggccaaccagcccgacagtgccatccccaccttcacctcggagggatggagaaagaagtggagacagtcctttcccaccattcctgcctttaagccaaagaaacaagctgtgcaggcatggtcccttaaggcacagtgggagctgagctggaaggggccacgtggatgggcaaagcttgtcaaagatgccccctccaggagagagccaggatgcccagatgaactgactgaaggaaaagcaagaaacagtttcttgcttggaagccaggtacaggagaggcagcatgcttgggctgacccagcatctcccagcaagacctcatctgtggagctgccacagagaagtttgtagccaggtactgcattctctcccatcctggggcagcactccccagagctgtgccagcaggggggctgtgccaacctgttcttagagtgtagctgtaagggcagtgcccatgtgtacattctgcctagagtgtagcctaaagggcagggcccacgtgtatagtatctgtatataagttgctgtgtgtctgtcctgatttctacaactggagtttttttatacaatgttctttgtctcaaaataaagcaatgtgttttttcggacatgcttttctgccactccatattaaaacatatgaccattgagtccctgctaaaaaaaaaaaaaaaaaaaaaaaaaa

SEQ ID NO: 2 - DNA, 1524 bp, Homo sapiens
gccagatctgtgagcccagcgctgactgcgccgcggagaaagccagtgggaacccagacccataggagacccgcgtccccgctcggcctggccaggccccgcgctatggagttcctctgggcccctctcttgggtctgtgctgcagtctggccgctgctgatcgccacaccgtcttctggaacagttcaaatcccaagttccggaatgaggactacaccatacatgtgcagctgaatgactacgtggacatcatctgtccgcactatgaagatcactctgtggcagacgctgccatggagcagtacatactgtacctggtggagcatgaggagtaccagctgtgccagccccagtccaaggaccaagtccgctggcagtgcaaccggcccagtgccaagcatggcccggagaagctgtctgagaagttccagcgcttcacacctttcaccctgggcaaggagttcaaagaaggacacagctactactacatctctcacagtcctcaggcccatgacaatccacaggagaagagacttgcagcagatgacccagaggtgcgggttctacatagcatcggtcacagtgctgccccacgcctcttcccacttgcctggactgtgctgctccttccacttctgctgctgcaaaccccgtgaaggtgtatgccacacctggccttaaagagggacaggctgaagagagggacaggcactccaaacctgtcttggggccactttcagagcccccagccctgggaaccactcccaccacaggcataagctatcacctagcagcctcaaaacgggtcagtattaaggttttcaaccggaaggaggccaaccagcccgacagtgccatccccaccttcacctcggagggatggagaaagaagtggagacagtcctttcccaccattcctgcctttaagccaaagaaacaagctgtgcaggcatggtcccttaaggcacagtgggagctgagctggaaggggccacgtggatgggcaaagcttgtcaaagatgccccctccaggagagagccaggatgcccagatgaactgactgaaggaaaagcaagaaacagtttcttgcttggaagccaggtacaggagaggcagcatgcttgggctgacccagcatctcccagcaagacctcatctgtggagctgccacagagaagtttgtagccaggtactgcattctctcccatcctggggcagcactccccagagctgtgccagcaggggggctgtgccaacctgttcttagagtgtagctgtaagggcagtgcccatgtgtacattctgcctagagtgtagcctaaagggcagggcccacgtgtatagtatctgtatataagttgctgtgtgtctgtcctgatttctacaactggagtttttttatacaatgttctttgtctcaaaataaagcaatgtgttttttcggacatgcttttctgccactccatattaaaacatatgaccattgagtccctgctaaaaaaaaaaaaaaaaaaaaaaaaaa

SEQ ID NO: 3 - DNA, 642 bp, Homo sapiens
atggcgcccgcgcagcgcccgctgctcccgctgctgctcctgctgttaccgctgccgccgccgcccttcgcgcgcgccgaggacgccgcccgcgccaactcggaccgctacgccgtctactggaaccgcagcaaccccaggttccacgcaggcgcgggggacgacggcgggggctacacggtggaggtgagcatcaatgactacctggacatctactgcccgcactatggggcgccgctgccgccggccgagcgcatggagcactacgtgctgtacatggtcaacggcgagggccacgcctcctgcgaccaccgccagcgcggcttcaagcgctgggagtgcaaccggcccgcggcgcccggggggccgctcaagttctcggagaagttccagctcttcacgcccttctccctgggcttcgagttccggcccggccacgagtattactacatctctgccacgcctcccaatgctgtggaccggccctgcctgcgactgaaggtgtacgtgcggccgaccaacgagaccctgtacgaggctcctgagcccatcttcaccagcaataactcgtgtagcagcccgggcggctgccgcctcttcctcagcaccatccccgtgctctggaccctcctgggttcctag

SEQ ID NO: 4 - DNA, 1798 bp, Homo sapiens
ggagctgggaagcggagaagccgggagcgcggggctcagtcggggggcggcggcggcggcggctccggggatggcggcggctccgctgctgctgctgctgctgctcgtgcccgtgccgctgctgccgctgctggcccaagggcccggaggggcgctgggaaaccggcatgcggtgtactggaacagctccaaccagcacctgcggcgagagggctacaccgtgcaggtgaacgtgaacgactatctggatatttactgcccgcactacaacagctcgggggtgggccccggggcgggaccggggcccggaggcggggcagagcagtacgtgctgtacatggtgagccgcaacggctaccgcacctgcaacgccagccagggcttcaagcgctgggagtgcaaccggccgcacgccccgcacagccccatcaagttctcggagaagttccagcgctacagcgccttctctctgggctacgagttccacgccggccacgagtactactacatctccacgcccactcacaacctgcactggaagtgtctgaggatgaaggtgttcgtctgctgcgcctccacatcgcactccggggagaagccggtccccactctcccccagttcaccatgggccccaatgtgaagatcaacgtgctggaagactttgagggagagaaccctcaggtgcccaagcttgagaagagcatcagcgggaccagccccaaacgggaacacctgcccctggccgtgggcatcgccttcttcctcatgacgttcttggcctcctagctctgccccctcccctggggggggagagatggggcggggcttggaaggagcagggagcctttggcctctccaagggaagcctagtgggcctagacccctcctcccatggctagaagtggggcctgcaccatacatctgtgtccgccccctctaccccttccccccacgtagggcactgtagtggaccaagcacggggacagccatgggtcccgggcggccttgtggctctggtaatgtttggtaccaaacttgggggccaaaaagggcagtgctcaggactccctggcccctggtacctttccctgactcctggtgccctctccctttgtccccccagagagacatatgcccccagagagagcaaatcgaagcgtgggaggcacccccattgctctcctccaggggcagaacatggggaggggactagatgggcaaggggcagcactgcctgctgcttccttcccctgtttacagcaataagcacgtcctcctcccccactcccacttccaggattgtggtttggattgaaaccaagtttacaagtagacacccctgggggggcgggcagtggacaaggatggcaaggggtgggcattggggtgccaggcaggcatgtacagactctatatctctatatataatgtacagacagacagagtcccttccctctttaaccccctgacctttcttgacttccccttcagcttcagaccccttccccaccaggctaggccccccacacctgggggaccccctggcccctcttttgtcttctgtgaagacaggacctatgcaacgcacagacacttttggagaccgtaaaacaacaacgccccctcccttccagccctgagccgggaaccatctcccaggaccttgccctgctcaccctatgtggtcccacctatcctcctgggcctttttcaagtgctttggctgtgactttcatactctgctcttagtctaaaaaaaataaactggagataaaaataaaaaaaaaaaaaaaaaa

SEQ ID NO: 5 - DNA, 1276 bp, Homo sapiens
cttccctcttcactttgtacctttctctcctcgactgtgaagcgggccgggacctgccaggccagaccaaaccggacctcgggggcgatgcggctgctgcccctgctgcggactgtcctctgggccgcgttcctcggctcccctctgcgcgggggctccagcctccgccacgtagtctactggaactccagtaaccccaggttgcttcgaggagacgccgtggtggagctgggcctcaacgattacctagacattgtctgcccccactacgaaggcccagggccccctgagggccccgagacgtttgctttgtacatggtggactggccaggctatgagtcctgccaggcagagggcccccgggcctacaagcgctgggtgtgctccctgccctttggccatgttcaattctcagagaagattcagcgcttcacacccttctccctcggctttgagttcttacctggagagacttactactacatctcggtgcccactccagagagttctggccagtgcttgaggctccaggtgtctgtctgctgcaaggagaggaagtctgagtcagcccatcctgttgggagccctggagagagtggcacatcagggtggcgagggggggacactcccagccccctctgtctcttgctattactgctgcttctgattcttcgtcttctgcgaattctgtgagccaagcagaccttccctctcatcccaaggagccagagtcctcccaagatcccctggaggaggagggatccctgctgcctgcactgggggtgccaattcagaccgacaagatggagcattgatgggggagatcagagggtctgaggtgactcttgcaggagcctgtcccctcatcacaggctaaagaagagcagtagacagccctggacactctgaagcagaggcaagacaaacacaggcgctttgcaggctgctctgagggtctcagcccatcccccaggaggactgggatttggtatgatcaaatcctcaagccagctgggggcccaggctgaagacctggggacaggtcgattgctggaccagggcaaagaagaagccctgccatctgtgccctgtgggccttttccctggggcagcaccttgccctccccaggggatcactcacttgtcttctatgaagacggactcttcatgaggttgaatttcatgccagtttgtatttttataagtatctagaccaaaccttcaataaaccactcatctttttgttgccctccccaaaaaaaaaaaaaaaaaaaaa

SEQ ID NO: 6 - DNA, 1110 bp, Homo sapiens
cttccctcttcactttgtacctttctctcctcgactgtgaagcgggccgggacctgccaggccagaccaaaccggacctcgggggcgatgcggctgctgcccctgctgcggactgtcctctgggccgcgttcctcggctcccctctgcgcgggggctccagcctccgccacgtagtctactggaactccagtaaccccaggttgcttcgaggagacgccgtggtggagctgggcctcaacgattacctagacattgtctgcccccactacgaaggcccagggccccctgagggccccgagacgtttgctttgtacatggtggactggccaggctatgagtcctgccaggcagagggcccccgggcctacaagcgctgggtgtgctccctgccctttggccatgttcaattctcagagaagattcagcgcttcacacccttctccctcggctttgagttcttacctggagagacttactactacatctcggtgcccactccagagagttctggccagtgcttgaggctccaggtgtctgtctgctgcaaggagaggagagccagagtcctcccaagatcccctggaggaggagggatccctgctgcctgcactgggggtgccaattcagaccgacaagatggagcattgatgggggagatcagagggtctgaggtgactcttgcaggagcctgtcccctcatcacaggctaaagaagagcagtagacagccctggacactctgaagcagaggcaagacaaacacaggcgctttgcaggctgctctgagggtctcagcccatcccccaggaggactgggatttggtatgatcaaatcctcaagccagctgggggcccaggctgaagacctggggacaggtcgattgctggaccagggcaaagaagaagccctgccatctgtgccctgtgggccttttccctggggcagcaccttgccctccccaggggatcactcacttgtcttctatgaagacggactcttcatgaggttgaatttcatgccagtttgtatttttataagtatctagaccaaaccttcaataaaccactcatctttttgttgccctccccaaaaaaaaaaaaaaaaaaaaa

SEQ ID NO: 7 - DNA, 1131 bp, Homo sapiens
cttccctcttcactttgtacctttctctcctcgactgtgaagcgggccgggacctgccaggccagaccaaaccggacctcgggggcgatgcggctgctgcccctgctgcggactgtcctctgggccgcgttcctcggctcccctctgcgcgggggctccagcctccgccacgtagtctactggaactccagtaaccccaggttgcttcgaggagacgccgtggtggagctgggcctcaacgattacctagacattgtctgcccccactacgaaggcccagggccccctgagggccccgagacgtttgctttgtacatggtggactggccaggctatgagtcctgccaggcagagggcccccgggcctacaagcgctgggtgtgctccctgccctttggccatgttcaattctcagagaagattcagcgcttcacacccttctccctcggctttgagttcttacctggagagacttactactacatctcggtgcccactccagagagttctggccagtgcttgaggctccaggtgtctgtctgctgcaaggagaggagaccttccctctcatcccaaggagccagagtcctcccaagatcccctggaggaggagggatccctgctgcctgcactgggggtgccaattcagaccgacaagatggagcattgatgggggagatcagagggtctgaggtgactcttgcaggagcctgtcccctcatcacaggctaaagaagagcagtagacagccctggacactctgaagcagaggcaagacaaacacaggcgctttgcaggctgctctgagggtctcagcccatcccccaggaggactgggatttggtatgatcaaatcctcaagccagctgggggcccaggctgaagacctggggacaggtcgattgctggaccagggcaaagaagaagccctgccatctgtgccctgtgggccttttccctggggcagcaccttgccctccccaggggatcactcacttgtcttctatgaagacggactcttcatgaggttgaatttcatgccagtttgtatttttataagtatctagaccaaaccttcaataaaccactcatctttttgttgccctccccaaaaaaaaaaaaaaaaaaaaa

SEQ ID NO: 8 - DNA, 1574 bp, Homo sapiens
gcttctctccatcttgtgattcctttttcctcctgaaccctccagtgggggtgcgagtttgtctttatcaccccccatcccaccgccttcttttcttctcgctctcctacccctccccagcttggtgggcgcctctttcctttctcgccccctttcatttttatttattcatatttatttggcgcccgctctctctctgtccctttgcctgcctccctccctccggatccccgctctctccccggagtggcgcgtcgggggctccgccgctggccaggcgtgatgttgcacgtggagatgttgacgctggtgtttctggtgctctggatgtgtgtgttcagccaggacccgggctccaaggccgtcgccgaccgctacgctgtctactggaacagcagcaaccccagattccagaggggtgactaccatattgatgtctgtatcaatgactacctggatgttttctgccctcactatgaggactccgtcccagaagataagactgagcgctatgtcctctacatggtgaactttgatggctacagtgcctgcgaccacacttccaaagggttcaagagatgggaatgtaaccggcctcactctccaaatggaccgctgaagttctctgaaaaattccagctcttcactcccttttctctaggatttgaattcaggccaggccgagaatatttctacatctcctctgcaatcccagataatggaagaaggtcctgtctaaagctcaaagtctttgtgagaccaacaaatagctgtatgaaaactataggtgttcatgatcgtgttttcgatgttaacgacaaagtagaaaattcattagaaccagcagatgacaccgtacatgagtcagccgagccatcccgcggcgagaacgcggcacaaacaccaaggatacccagccgccttttggcaatcctactgttcctcctggcgatgcttttgacattatagcacagtctcctcccatcacttgtcacagaaaacatcagggtcttggaacaccagagatccacctaactgctcatcctaagaagggacttgttattgggttttggcagatgtcagatttttgttttctttctttcagcctgaattctaagcaacaacttcaggttgggggcctaaacttgttcctgcctccctcaccccaccccgccccacccccagccctggcccttggcttctctcacccctcccaaattaaatggactccagatgaaaatgccaaattgtcatagtgacaccagtggttcgtcagctcctgtgcattctcctctaagaactcacctccgttagcgcactgtgtcagcgggctatggacaaggaagaatagtggcagatgcagccagcgctggctagggctgggagggttttgctctcctatgcaatatttatgccttctcattcagaactgtaagatgatcgcgcagggcatcatgtcaccatgtcaggtccggaggggaggtattaagaatagatacgatattacaccatttcctataggagtatgtaaatgaacaggcttctaaaaggttgagacactggttttttttttt

SEQ ID NO: 9 - DNA, 3346 bp, Homo sapiens
gagtagacagcacagcggcagcggagggagtctatgcgagctggacagcagtgggaggtttgtgaggctcgcactggccgcagaccctcgggctcgatcgcccgggagccaggactcggcgacgcgaggctgccgggctacccggccgaggcttcgggggcgcaaactaatgggactggctcgctcggcagcatctccccgctcttctaagtacactgagcagggcccgcgctgaagtagaagctgtccgggggcgcgtagcccggagtcccagtgtggcccggaggaacggagcccgtgccagggcggcccagtcgggagcccggggaccgagcttgtgctgtggggaaacccccacttcttccaagggacagcgatcccgggacggtcgaggcgtcggggcggtcaccgagacctctgcgggaagaccccgtcggggagagggcgcgcagccccgaagcgtctcgggaagtcgagcggaatcgggcgggatcacccgggggcgcagagcccccgtcgcgcctcgtgcggcagcggagagcccaggagaacgagccctcgggggccgaagcccatgcccgggttgggggcggctgcccagtgagtcctcctggccggccgggcggagaagagcgacaccgaagccggcgggaggggagcacttcaaggccggcggctgcggaggatgggcgcctgagcggctccgagcgcagcgcggcagaggaaggcgaggcgagctttggtgaggaggcgccaagggatcccgaagtgcagtctgcccccgggaagatggctcggcctgggcagcgttggctcggcaagtggcttgtggcgatggtcgtgtgggcgctgtgccggctcgccacaccgctggccaagaacctggagcccgtatcctggagctccctcaaccccaagttcctgagtgggaagggcttggtgatctatccgaaaattggagacaagctggacatcatctgcccccgagcagaagcagggcggccctatgagtactacaagctgtacctggtgcggcctgagcaggcagctgcctgtagcacagttctcgaccccaacgtgttggtcacctgcaataggccagagcaggaaatacgctttaccatcaagttccaggagttcagccccaactacatgggcctggagttcaagaagcaccatgattactacattacctcaacatccaatggaagcctggaggggctggaaaaccgggagggcggtgtgtgccgcacacgcaccatgaagatcatcatgaaggttgggcaagatcccaatgctgtgacgcctgagcagctgactaccagcaggcccagcaaggaggcagacaacactgtcaagatggccacacaggcccctggtagtcggggctccctgggtgactctgatggcaagcatgagactgtgaaccaggaagagaagagtggcccaggtgcaagtgggggcagcagcggggaccctgatggcttcttcaactccaaggtggcattgttcgcggctgtcggtgccggttgcgtcatcttcctgctcatcatcatcttcctgacggtcctactactgaagctacgcaagcggcaccgcaagcacacacagcagcgggcggctgccctctcgctcagtaccctggccagtcccaaggggggcagtggcacagcgggcaccgagcccagcgacatcatcattcccttacggactacagagaacaactactgcccccactatgagaaggtgagtggggactacgggcaccctgtctacatcgtccaagagatgccgccccagagcccggcgaacatctactacaaggtctgagtgcccggcacggcctcaggcccccgagggacagtcggcctggaccggacctctcctttcgcccccacaccccctccccttgccagctgtgcccacctttgtatttagttttgtagtttcttggcttttataatccccctttttccctgccccctgggcttcggaggggggtgcttgtgcccctaacccccatgctcttgtgccttccccctctggccaggcctctgggctccgtgggggcgccccttcttggaaggcagggctggacactgatggacagcaggcagggagacagtcccctggccctgcccctccctcgccccccttgccaccttcccaggactgcttgtccgctatcatcactgtttttaatgcttttgtgttcattttttagctgtcaactcattttcatctgttttttgaagaaaaatggaaaaatgtaaaaggcagcccctccccaggctttgtgagcctggcccaagccagtacaagagggcctggggcacgatgtggtcagccaggaagcataggatgccatttcttttatagattccttggtatttctggtggggtaaggggcaggccagggctgttcacgcccatgagggaagaggaaagtgccactgggcaaggtgtcccaccctcccctcctgaccctcctacgaggcttatcctggcaatggggtagtcactgccacccttccacacacacacacacacacacacacacaaaaaaaaatcccttccttgtgggattcttgggcatctcctgcctccctcactctcacggtaattaatgtcttaattggctgttgcctggggaacaggagagctgctgcaggcagatgacctcatggggggtggagggaggtgaggtgcccaggtggctatttgccctgcagagctgggagtttcacccccaccccccaccctgttctctccttacctttggcatcctttggcctggtggggaaacagaggcccagggtggagacctaagcgggtataagaccaggtggcctgctccttttctgggccctagcacaggtgggtaacccccacccaacccagctcctgctgctgtcccagtcttgggctggggcctggaaagaggaagaggctgcctggggctgggccagcccgctgtgcactttgaccccagttccttgccagcacggctgctaacagactgccacttgagtgcgccttgcaggcactcccagagcagccatggaaggagctggccctcacaccatccacctccacactgcctcctggccagctgcccaccccagtgccaggtgggagagggagcagaacagccagccccttccaggtggcagtcggaagggtttttgtttttgtttctgttgccatttgtgtaaatactagtctttttggaaaaaaaataatgtaaagatgttttgtataaactctgaattattttcttgttgcttttttcttagaaaaaaatgagaactaaaaaaaaaaaattaaccacatggaaaaaaaaaaa

SEQ ID NO: 10 - DNA, 4335 bp, Homo sapiens
gcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaaaaaaaaaaaaaaa

SEQ ID NO: 11 - DNA, 3236 bp, Homo sapiens
ggtttcctcccttagcccgctgccctcaatcccagcgaggctggggctccggctcggcgcccccttcctcgctccctggtccggcgccccatgccgcccccgcccggtccccggctcccccagtcccccacttaggcgggctcacagatcccggggtgctggcgcgtgggccgggggcgcgtagggcgcctgcagacggcccctggaagggctctggtggggctgagcgctctgccgcgggggcgcgggcacagcaggaagcaggtccgcgtgggcgctgggggcatcagctaccggggtggtccgggctgaagagccaggcagccaaggcagccaccccggggggtgggcgactttgggggagttggtgccccgccccccaggccttggcggggtcatggggcccccccattctgggccggggggcgtgcgagtcggggccctgctgctgctgggggttttggggctggtgtctgggctcagcctggagcctgtctactggaactcggcgaataagaggttccaggcagagggtggttatgtgctgtaccctcagatcggggaccggctagacctgctctgcccccgggcccggcctcctggccctcactcctctcctaattatgagttctacaagctgtacctggtagggggtgctcagggccggcgctgtgaggcaccccctgccccaaacctccttctcacttgtgatcgcccagacctggatctccgcttcaccatcaagttccaggagtatagccctaatctctggggccacgagttccgctcgcaccacgattactacatcattgccacatcggatgggacccgggagggcctggagagcctgcagggaggtgtgtgcctaaccagaggcatgaaggtgcttctccgagtgggacaaagtccccgaggaggggctgtcccccgaaaacctgtgtctgaaatgcccatggaaagagaccgaggggcagcccacagcctggagcctgggaaggagaacctgccaggtgaccccaccagcaatgcaacctcccggggtgctgaaggccccctgccccctcccagcatgcctgcagtggctggggcagcaggggggctggcgctgctcttgctgggcgtggcaggggctgggggtgccatgtgttggcggagacggcgggccaagccttcggagagtcgccaccctggtcctggctccttcgggaggggagggtctctgggcctggggggtggaggtgggatgggacctcgggaggctgagcctggggagctagggatagctctgcggggtggcggggctgcagatccccccttctgcccccactatgagaaggtgagtggtgactatgggcatcctgtgtatatcgtgcaggatgggcccccccagagccctccaaacatctactacaaggtatgagggctcctctcacgtggctatcctgaatccagcccttcttggggtgctcctccagtttaattcctggtttgagggacacctctaacatctcggccccctgtgcccccccagccccttcactcctcccggctgctgtcctcgtctccacttttaggattccttaggattcccactgccccacttcctgccctcccgtttggccatgggtgcccccctctgtctcagtgtccctggatcctttttccttggggaggggcacaggctcagcctcctctctgaccatgacccaggcatccttgtccccctcacccacccagagctaggggcgggaacagcccaccttttggttggcaccgccttctttctgcctctcactggttttctcttctctatctcttattctttccctctcttccgtctctaggtctgttcttcttccctagcatcctcctccccacatctcctttcaccctcttggcttcttatcctgtgcctctcccatctcctgggtgggggcatcaaagcatttctccccttagctttcagccccccttctgacctctcataccaaccactcccctcagtctgccaaaaatgggggccttatggggaaggctctgacactccaccccagctcaggccatgggcagcagggctccattctctggcctggcccaggcctctacatacttactccagccatttggggtggttgggtcatgacagctaccatgagaagaagtgtcccgttttgtccagtggccaatagcaagatatgaaccggtcgggacatgtatggacttggtctgatgctgaatgggccacttgggaccggaagtgacttgctccagacaagaggtgaccaggcccggacagaaatggcctgggaagtagcagaagcagtgcagcaggaactggaagtgccttcatccaggacaggaagtagcacttctgaaacaggaagtggtctggctggaactccaagtggcttagtctgggggatcaggaggtgggaggtggatggttcttattctgtggagaagaagggcgggaagaacttcctttcaggaggaagctggaacttactgactgtaagaggttagaggtggaccgagaaggacttttcccagtcttcagtggcacttcccaagatctcccttcccttgtgctctgtgctgattttaggacagctaagatgactgccatgtgctgtggcaggcctaatttgtcttgttctttcctttccatatcccagtataatctctgttaatcaacaggactaccccaagaacccatgtgctctcccgagtaacccagatggctgtcttgttcattccatcctacatttctgactcctttcagactcaacacagttcccttcttagtgaccaaaatggtggcctactggctggtctagctgacagtggtacttagcaaaggccactgtttccatagtgaccagctgatacctcttcctgccctctagtgtgcaattgggtgttgcctcagtttcctcccagctcagttttattagatcaaagctgttgttgggcaccaggttggccacctcaatcaccagccaagatggttgctttgtccaccagaggtcaagttcacctctctggtgctgtagttcccagctccttcctgatttttctaatcgctccttctggggaacaggaagttgatattgccatggtggcggggtatgccgtcacctcagtagttttactgtaaaagggaaatttgaacaacaaaaaccaaaaaaaataaaaataaaaaacttcaaaagttgacaaaaaaaaaaaaaaaaaaa

SEQ ID NO: 12 - DNA, 1474 bp, Mus musculus
gagatttgtgatcgtggtgcctactgtaccgtggagaagcctgtgggaaccttacactgcagagctctcgtcctggcctggctcggcccggccccgcgctatggagttcctttgggcccctctcttgggtctgtgctgcagtctggccgctgctgaccgccacatcgtcttctggaacagttcaaatcccaagttccgtgaggaggactacacggtgcacgtgcagctgaatgactacctagacatcatctgcccacattacgaggacgactctgtggcagatgcagccatggagcgatacacactgtacatggtggaacaccaggagtatgtggcatgccaaccccagtccaaggaccaggtccgttggaattgcaaccggcccagtgccaagcatggcccggagaagctgtctgtgaaattccagcgcttcacgccttttatcttgggcaaggagttcaaggaaggacacagctactactacatctccaaacctatctaccatcaggaatcccagtgcttgaagctgaaggtgactgtcaatggcaaaatcactcataatccccaggcccatgtcaacccacaggagaagagactccaagcagatgacccggaagtacaggttttgcacagcattggttacagtgccgccccccgcctcttcccactggtctgggcagtattgctcctaccactgctgctgctgcaatctcagtgagggtgtacgcttgccctggcttatggattggaatgggactaaggggcagccccagccctgggaacctctcccactacacccacaagacgccaccatgaagcctcaaaaggttcagtattaagggttttaaccgaaaagagtttaaccagcccaactgtgccatccctgcctccacttcagagggatggagaaagaagtggagaaggtccttaacctgcagtttctgcctttaagccaaagaaacaagctgtgcggacctggcccattaagaggcctcagtgggagaagggctaaaaagagcctgaggtcatgcggttggacactgaacttggtaaagacgtccttccccaaggaagccataatgtgcagatgaactgaccgaaggaaaagcttgagacagcttcctgcgggggagccaggtacagaagaggcagcttgggctgacccagcatctctgcaagatttccacctgctgggctgccagagaggtttgtagcccggccctgactgcatcctccccatccctggggcaacattccctggagctgtgccaacaggaggactgaagcagcctggttttagagtgtagctgtaagggcagtgcccgtgtgtacagtctgtggagtttagcttaacgggcagggcccacatgtacagtgtctgtatataaatgtgctgtatctgttcatttctatgactgcagggtttttttatacagtgtttttggattcataataaattgatggttttttatgg

SEQ ID NO: 13 - DNA, 1766 bp, Mus musculus
ggggagcccccgcgcccggggaacccgggagagcgccttgcgcagtgcaccgctcgggcctctgccccgcgccgccgtcgccgccgccgccgccgccgccgccgccgcctggtttctcggcgcccgcggtctacggcgggggccgcgcgcagaggcggggcggcagatctccaagcgcgcgcggcgctccgcccgctcggcggcggaggcggaggcggagacggcggggccaggcggcggctggagagcgagggagcgagcgcggcggcagcaccggggccatggcgcccgcgcagcgcccgctgctgccgctgctgctgctgctgctgccgctgcgtgcgcgcaacgaggacccggcccgggccaacgctgaccgatacgcagtctactggaaccgtagcaaccccaggtttcaggtgagcgctgtgggtgatggcggcggctataccgtggaggtgagcatcaacgactacctggatatctactgcccacactacggggcgccgctgcccccggctgagcgcatggagcggtacatcctgtacatggtgaatggtgagggccacgcctcctgtgaccaccggcagcgaggcttcaagcgctgggaatgcaaccggcccgcagcgcccgggggacccctcaagttctcagagaagttccaactcttcacccccttttccctgggctttgagttccggcctggccacgaatactactacatctctgccacacctcccaacctcgtggaccgaccctgccagcgactcaaggtttatgtgcgtccaaccaatgagaccctgtatgaggctccagagcccatcttcaccagtaacagctcctgcagcggcctgggtggctgccacctcttcctcaccaccgtccctgtgctgtggtcccttctgggctcctagtgtcaggccggagaacaccagccccacctggaccccgtgacctttgccctctgacctgccacggccacctccgagacaaaatccttgctgcttctctttcatggtgctgtcccgccggaggaggccatccatccgtccctgggatgcaacatggggtcccaatgcctgaggagaagacccccccccaaggctgactcgctttcaccagggccaccagggccatccagtgttgtttaattacagtcggaaagacttaaggtttttcttttaatttaatttattccctgacattgctggtgacactgggaagggaacaagccacagggatgaggtgaagccatctctgtccttcctggaataccggagatccaggggcctccagctgctcctttcttctgtgtcctgttatttgggtcccagatggagcccaccgcggacttgccttgcattcctcaggccaggcaagcctgagccagaaagggcgcacggtgccagcccctctcggggactctgggggtgccatcccccactcttcttccagccactctcgggccccactcccacatcatctcagaaacccttcagccctcgcaactcgcccctccgggcccccccaccaggcacaaccatccccggggccagccgggacgttgtcggtttatttctgtaaatagaaaccagcaagtgtatactgtgatttattttaatgtattcttaaggacagaatggaaattctttaaaaaaaattttttttccgaccttcaattcaaggggtcatttattttggtggggggagtggggtggactttttaggatagaagcaacactttgcaataaactcatttttttttgttc

SEQ ID NO: 14 - DNA, 699 bp, Mus musculus
gaagggcggagggaaggattgtgggcgtgactaagattgggggctggcagcggcggcagcagggagctgggaagctgagaagccgggagtgcggggctcagtcggggggcggcggcggcggcggctccggggatggcggcggctccgctgctgctgctgctgctgctcgtgcccgtgccgctgctgccgctgctggcccaggggcctgggggcgcactgggaaaccggcatgcggtatactggaacagctccaatcagcacctgcggcgagagggctacaccgtgcaggtgaacgtgaacgactatctggatatttactgtccgcactacaacagctcagggcctggcggcggggcggagcagtacgtgctgtacatggtgaacctgagcggctaccgcacctgcaacgccagccaaggctccaagcgctgggaatgcaaccggcagcacgcctcgcacagccccatcaagttctccgagaagttccagcgttacagcgccttctcgctgggctatgaattccatgccggccaagaatactactacatctccacgcccactcacaacctgcactggaagtgtctgaggatgaaggtgttcgtctgctgcgcctccacatcgcactccggggagaagccggtccccactctcccccagttcaccatgggccccaatgtgaagatcaacgtgttgggttag

SEQ ID NO: 15 - DNA, 2363 bp, Mus musculus
gaagggcggagggaaggattgtgggcgtgactaagattgggggctggcagcggcggcagcagggagctgggaagctgagaagccgggagtgcggggctcagtcggggggcggcggcggcggcggctccggggatggcggcggctccgctgctgctgctgctgctgctcgtgcccgtgccgctgctgccgctgctggcccaggggcctgggggcgcactgggaaaccggcatgcggtatactggaacagctccaatcagcacctgcggcgagagggctacaccgtgcaggtgaacgtgaacgactatctggatatttactgtccgcactacaacagctcagggcctggcggcggggcggagcagtacgtgctgtacatggtgaacctgagcggctaccgcacctgcaacgccagccaaggctccaagcgctgggaatgcaaccggcagcacgcctcgcacagccccatcaagttctccgagaagttccagcgttacagcgccttctcgctgggctatgaattccatgccggccaagaatactactacatctccacgcccactcacaacctgcactggaagtgtctgaggatgaaggtgttcgtctgctgcgcctccacatcgcactccggggagaagccggtccccactctcccccagttcaccatgggccccaatgtgaagatcaacgtgttggaagactttgagggagagaatccccaggtgcccaagcttgagaagagcatcagtgggaccagccccaagcgggaacacctgcctctggccgtgggcatcgccttcttcctcatgacgctcttggcctcctagctctgccccttcctatgacagagagaggtgggaagggctgagaaggagcagggagcttgctgtggggcctacatccttcttcccatggttgggagcggggtctgcactgtacatctctctgggcctgccctctttgcccacacactctctaggacaggcaccgtagtggatcaggcacagggacagccacaggtcccaggtggcctgtggctttggtaatgtttggtaccaaacctgggggctataaaggcagtgctcaggactccctggcccctggtacctttccctgacccttggtgccctccctctttgtcccccaagaggcaaatatgccccagagagaacaaacgaagcatgggaggtgccccctgtcctctcctctggggcagaacatggggaggggactaggtgggtgaggggtggagcctcaggctgcccctccccctgtttacagcaataagcatgtcctctccctccactcccacatagaggactgtggtttggattgaatccaagtttacaaatagacacccctgggggggagcaggcagtggacaggggtgggcatcggggtgccagacaggcatgtacagactctatatctctatatataatgtacagacagacagagtcccttccccttcttaaccttgacctttctcgacttccccttccagactcagaccccttcccctaccaggttaggccccccttgggacccccctggcccctcttttgtcttctgtgaagacaggacctatgcaacgcacagacacttttggagaccgtaacaacaatgccccctcccttccagccctgagccgggaaccaactcccaggaccttgccctgcccaccctaagtggtccccacccatcctgggcctttttcaagtgctttggctgtgactttcatactctgctcttagtctaaaaaaaataaactggagataaaaatacctgagtgtgtgtggtttcaaggggccacgtccctctgtttcactggctgaaccaatcctgtggggctcaaggcccgggtgagcaggcattctaggttagaggccacagagtggagagagggacagagcttcaagttgagcccgggtgactttgggcaggttccttaacctttctaggctccggcttcccacagggaaagtggtgtgagtacccgcttcccaggccctgtgtacccgagttaaggctcaggctggtgcgctctatgctgtagtcagaagcttttggctgggtccagggatttcaggggatttgtgatcctagataggaaaaaaaaagttacatggttattttcactcacctgtgacaagtttaacattccctttctgtacttgggcaacaggccacagcagcataaagagtacgtgtgactttgtagccaacagaaatcccagatgttttcagacctgctttatatctcagaatatcatttatgtgtcactactttgaaatgatcatagttgttagactttacttgtttttaacatgttaataaagaaacatatatattacataccaaaaa

SEQ ID NO: 16 - DNA, 813 bp, Mus musculus
gagacctgggcatcttctggcttaatgctgctgagacttgggttgatatatcctccaacacggccccccgccccaccgggcccgctggttcccctgctgcggactgtgctctgggccgcgctgctcggctcgcgcctgccggggtgctccagcctccgccacccaatctactggaactcctctaaccccaggttgctccgaggagatgccgtggtggagctgggcttcaacgattacctagacatcttctgcccacattatgaaagcccagggcccccagaaggcccggaaacctttgcattatacatggtggactggtcaggctacgaggcctgcacggcagagggggcaaatgccttccagcgctggaattgctcgatgccttttgcccctttcagccctgttcgattctcagaaaagattcagcgctacacacccttcccgctgggctttgagttcttgcctggagagacttactactacatctcggtgccgactccggagagtcctggccggtgcctgagactccaggtgtctgtctgctgcaaggagagcgggtcatcacatgagtcagctcatcctgttgggagtcctggagaaagcggtacgtccgggtggcggggaggacacgcgcccagccccctgtgtctcttgctgctgctgctgctcccaatcctccgtctcctgagagttctgtgagccaagcacaccttccaagtcaccccaaggaaccgcagcccttgtcaagagcctctgtggaaggggttcctgccaccatacagcgatgccaaaccaggcagacaggatgaaact

SEQ ID NO: 17 - DNA, 3473 bp, Mus musculus
cctccttcttctctctctcctcttcctcttcctcccgcgctcgccgcgcgcctctcgccccttcatcgtatttattcctatttatttggcgtccgctctcttcgtctgcccctctccctccctccccctggatccccgatctctccccggagtggcgcgtcgggggctccgccgctggctaggcgtgatgttgcacgtggagatgttgacgctgctctttctggtgctctggatgtgtgtgttcagccaggacccgggctccaaagtcgtcgccgaccgctacgccgtctactggaacagcagcaaccccagattccagaggggtgactaccacattgatgtctgtatcaatgactacctggatgttttctgccctcactatgaggactctgtcccagaagacaagactgagcgctacgtcctgtacatggtgaattttgatgggtacagtgcctgcgaccacacgtccaaagggttcaagagatgggaatgtaaccggcctcactccccaaacggaccgctgaagttctcggaaaaattccagctcttcactcccttttctttaggatttgaattcaggccaggccgagagtatttctacatctcctctgcaatcccagacaacggaagaaggtcctgtctaaagctcaaagtctttgtgagaccaacaaatagctgtatgaaaactataggtgttcatgatcgtgttttcgatgttaacgacaaagtagaaaattcattagaaccagcagatgacaccgtacatgagtcagccgagccatcccgcggtgagaacgcggcgcagacaccaaggatacccagccgccttttggcaatcctactgttcctcctggcgatgcttttgacattatagcacagtctcctcccgtcgcctgtcacagaaaatgtcagggtcttggaacaccagagatccacctaactgctcatcccaagaagggacttgttattggtttttggcagatgtcagatttttgttttctttccttcagcctgatttctaagcaacaacttggggttgttgggtgggtggggggggggtaaaccagttgctgcctccttcaccccaccctaccctaagcccttgcccttgactcctctcatccccttcccaatcaaacggagtccagataaaaatgccagattgtcagagtgacagcagcaggtcttggcctcctgtgcgttttctagagctcacctccttaggttcagggtatccaccatatgcacacaagaaagaatggcggcagatgctgcctgcaatgtccaggcctgagaggggtgccctctcctatacaatacttatgccttcacggtcaggacagtaaggtgattgtgtggagcttggtgttggcctgtcgagtccagagggggcggagttaagaataggcctaatactatcccacatcctctaggagtgtggaagtgagcaggctttgcaaaggttcagatactagtttttatttcatcaaatgggactgtcttaaagcattttttttttttttttttttttttgccagcaaaacttgggctgtgttgaagaagccttttctcccaggaggaaggttggatgatgcagggatgctaataagaggaagtgtggatattgagaaaatgatctgttgctggaggtgtgagtgcctctaactttttaggaccgggcagtgcacacctgagggtttcttttcttttctttttttttttaaatctttgaatacagttcaccatggtgctacaactttttttttctttttaggacttcaaagaggcatttttatgaataaagtgaccttccccaaggctgccaagccagggttgatgagtagagagtggaatagctttggctactcctctggggacaatatgtacaaggaaagagagtctgaccagaggagatgcaatttggctttgctggagctccagtgtgctgtggcctttccccaattcccaccccgatacccacgttctgctccttgctccgtgaccacaggggactcagacacagacccctatcaccaggagagtcagtcagcactacttgggagggctaaagggaaatttggaatacaaattccaaattttggaataagaaattcaagtgttgatgttctatcctttccctttctgacacagcctaaagcgtagggggaaacgcgtgtttatctgtgggagataaacaagatggagtcccaaagactttgacaaaatatttttttaaaaatccactagaatagaaaatacattatttagatatactttatgctgagagtgagtatatatgcttgtcctatttaaacttgtgagaaaaagtggtatcccttggtacatttagaaacacagggggttatcttgtttcatagtggggtttggggcagaagaatgaacatgggatgaccctgttgaaagaaacttagcatggccgaggggttggttccagctgattgccaggaaacacaagcagtgggggtctccttgtggggtgtcgggagctttaaaccccaaagccaagcgagacagggaatagtgtcagaccaatggaaaagtgatctagcctatttttgctattgcacgacatcaatagaaaccatgcctttgtaaacaagcaagtcagtaaatgcaatgatcatgtaatgtggaaaattgaaagcattccagtttaaaaaatatggaattttttatatattcgtttttaagatgtatatgtaaaaaaaagagttcacattatagatggacttcatggatcggaaattgcttagaatttcgagtggtttcaaattagaaaaatatgtgagtggaaggcagctgtgaacgaactgcaccctagagagttgtacgcacgttggaatgtaagctgggcttacttacctggtccctttggagtggaggcaactggagtctgctctcacctgaaccatgtgaccggagtcgccagcctcaaagatatagttagtcctctcccctcttctcccaacatctgagacttctctggagtagcagagtctcatcacagaaggcacccaacatttccactgaaacagaacttcaaagcattggatttctttgcctttatttatgcgctcgacactctcagtgatatccagatgtattctttttattaatagttacaggtttgtgggttagtgggatttcagtaaagggacaaaaaaaaaaatcccttctaagatggatcgatagaaccaagataatactgcatgtactacagtcaaggacaccaagtgatcctatggtgattccagttagtcctaacgacattagtggttctaatgtcattgtagaaatggtgagtcctgtgattctagcatttgtctctaaccaatgatgaaacaattcctcatggccctctctgccattgtctttcatttcccactgtgcaattagacccttttacttcactgccatgccaccaattattaagtttaaaaaaaaaaaaaaaa

SEQ ID NO: 18 - DNA, 3392 bp, Mus musculus
cctccttcttctctctctcctcttcctcttcctcccgcgctcgccgcgcgcctctcgccccttcatcgtatttattcctatttatttggcgtccgctctcttcgtctgcccctctccctccctccccctggatccccgatctctccccggagtggcgcgtcgggggctccgccgctggctaggcgtgatgttgcacgtggagatgttgacgctgctctttctggtgctctggatgtgtgtgttcagccaggacccgggctccaaagtcgtcgccgaccgctacgccgtctactggaacagcagcaaccccagattccagaggggtgactaccacattgatgtctgtatcaatgactacctggatgttttctgccctcactatgaggactctgtcccagaagacaagactgagcgctacgtcctgtacatggtgaattttgatgggtacagtgcctgcgaccacacgtccaaagggttcaagagatgggaatgtaaccggcctcactccccaaacggaccgctgaagttctcggaaaaattccagctcttcactcccttttctttaggatttgaattcaggccaggccgagagtatttctacatctcctctgcaatcccagacaacggaagaaggtcctgtctaaagctcaaagtctttgtgagaccaacaaatgacaccgtacatgagtcagccgagccatcccgcggtgagaacgcggcgcagacaccaaggatacccagccgccttttggcaatcctactgttcctcctggcgatgcttttgacattatagcacagtctcctcccgtcgcctgtcacagaaaatgtcagggtcttggaacaccagagatccacctaactgctcatcccaagaagggacttgttattggtttttggcagatgtcagatttttgttttctttccttcagcctgatttctaagcaacaacttggggttgttgggtgggtggggggggggtaaaccagttgctgcctccttcaccccaccctaccctaagcccttgcccttgactcctctcatccccttcccaatcaaacggagtccagataaaaatgccagattgtcagagtgacagcagcaggtcttggcctcctgtgcgttttctagagctcacctccttaggttcagggtatccaccatatgcacacaagaaagaatggcggcagatgctgcctgcaatgtccaggcctgagaggggtgccctctcctatacaatacttatgccttcacggtcaggacagtaaggtgattgtgtggagcttggtgttggcctgtcgagtccagagggggcggagttaagaataggcctaatactatcccacatcctctaggagtgtggaagtgagcaggctttgcaaaggttcagatactagtttttatttcatcaaatgggactgtcttaaagcattttttttttttttttttttttttgccagcaaaacttgggctgtgttgaagaagccttttctcccaggaggaaggttggatgatgcagggatgctaataagaggaagtgtggatattgagaaaatgatctgttgctggaggtgtgagtgcctctaactttttaggaccgggcagtgcacacctgagggtttcttttcttttctttttttttttaaatctttgaatacagttcaccatggtgctacaactttttttttctttttaggacttcaaagaggcatttttatgaataaagtgaccttccccaaggctgccaagccagggttgatgagtagagagtggaatagctttggctactcctctggggacaatatgtacaaggaaagagagtctgaccagaggagatgcaatttggctttgctggagctccagtgtgctgtggcctttccccaattcccaccccgatacccacgttctgctccttgctccgtgaccacaggggactcagacacagacccctatcaccaggagagtcagtcagcactacttgggagggctaaagggaaatttggaatacaaattccaaattttggaataagaaattcaagtgttgatgttctatcctttccctttctgacacagcctaaagcgtagggggaaacgcgtgtttatctgtgggagataaacaagatggagtcccaaagactttgacaaaatatttttttaaaaatccactagaatagaaaatacattatttagatatactttatgctgagagtgagtatatatgcttgtcctatttaaacttgtgagaaaaagtggtatcccttggtacatttagaaacacagggggttatcttgtttcatagtggggtttggggcagaagaatgaacatgggatgaccctgttgaaagaaacttagcatggccgaggggttggttccagctgattgccaggaaacacaagcagtgggggtctccttgtggggtgtcgggagctttaaaccccaaagccaagcgagacagggaatagtgtcagaccaatggaaaagtgatctagcctatttttgctattgcacgacatcaatagaaaccatgcctttgtaaacaagcaagtcagtaaatgcaatgatcatgtaatgtggaaaattgaaagcattccagtttaaaaaatatggaattttttatatattcgtttttaagatgtatatgtaaaaaaaagagttcacattatagatggacttcatggatcggaaattgcttagaatttcgagtggtttcaaattagaaaaatatgtgagtggaaggcagctgtgaacgaactgcaccctagagagttgtacgcacgttggaatgtaagctgggcttacttacctggtccctttggagtggaggcaactggagtctgctctcacctgaaccatgtgaccggagtcgccagcctcaaagatatagttagtcctctcccctcttctcccaacatctgagacttctctggagtagcagagtctcatcacagaaggcacccaacatttccactgaaacagaacttcaaagcattggatttctttgcctttatttatgcgctcgacactctcagtgatatccagatgtattctttttattaatagttacaggtttgtgggttagtgggatttcagtaaagggacaaaaaaaaaaatcccttctaagatggatcgatagaaccaagataatactgcatgtactacagtcaaggacaccaagtgatcctatggtgattccagttagtcctaacgacattagtggttctaatgtcattgtagaaatggtgagtcctgtgattctagcatttgtctctaaccaatgatgaaacaattcctcatggccctctctgccattgtctttcatttcccactgtgcaattagacccttttacttcactgccatgccaccaattattaagtttaaaaaaaaaaaaaaaa

SEQ ID NO: 19 - DNA, 3261 bp, Mus musculus
gatgcgcgctggacagcagtggggggtgtgtgtgattcgcactgactgcagacactcgggctcaatcgccggggattaaggacttggcgaccgggctgccgggctacccggccgaggcttcagaggtgcaaatcgacgggactgatttgctcggcagggccgtactagccttttaagtatactgagaagggccggtgctaaagtagaagctgtcgtgggacgcgccggcgcggagtcccactgtggcccggaggaacggagtccgtgttagggggacccagtcgggagcctggggaccgagctcgcattgttgggagacaacccgcctcttcttcccagaggcgtcaatcccaggacggtcgaggcgtcggggcagccactgagcgctcttcagggagacccgatcggggagaggacttgcagtctcaaacaatctgggagagccgagcggaatccggcgggatcactcgggggcggcgagcccccgtcgcgcctcgtgcggcggagaggccagggaagaaagtcctcggaggccgcagcccatgcgcgggttggggacgcctgcccagtgagtcctcctggccagccgggcgaaggagagcgaccccgaggccagcgggaggggagtgcttcaaggccggcggctgcggaggatgggcgcctgagtggctccgagcgctgcgcggcacgggcaggcgaggcgagctttgctgaggaggcgctaggggatcctgaagtgcattctgccccggggaaaatggcccggcctgggcagcgttggctcagcaagtggcttgtggctatggtcgtgctgacgctgtgccggcttgccacgccgttggccaagaacctggagcccgtgtcctggagctctcttaaccctaagttcctaagtgggaagggcttggtgatctacccgaagattggagacaagctggacatcatctgcccccgagcagaagcagggcggccctacgagtactacaagctgtacctggtgcggccagagcaggcggctgcttgcagcactgtgcttgatcccaatgtactggtcacttgcaacaagccacaccaggaaatccgcttcaccatcaagttccaagagttcagccccaactacatgggcctggaattcaaaaagtaccacgattactacattacatcaacgtccaatgggagcttggagggactggagaaccgggagggaggtgtgtgtcgcacccgcactatgaagatcgttatgaaggttgggcaagatccaaatgctgtgacacccgagcagttgactaccagccggccaagcaaggagtcagacaacactgtcaagacagccacacaggctcctggtcggggatcccagggtgactctgacggcaagcatgagactgtgaaccaggaagagaagagtggcccaggtgcaggtggcggtggcagcggggactctgacagcttcttcaactccaaggtagcattgttcgcagccgtcggcgccggctgtgtcatcttcctgctcatcatcatcttcttgacagtcctactactcaagctccgcaagcgccatcgcaagcatacacagcagcgggcggctgccctctcgctcagtaccctagccagccccaaagggggtagtggtacagcgggcaccgagcccagcgacatcatcatccccttacggactacagagaacaactactgcccccactatgagaaggtgagtggggactacgggcatcctgtctacatcgtccaggagatgccccctcagagcccggcgaacatctactacaaggtttgaaggcccagcatggcctcaggccccacaggacacatggcctggatcggccgctctcttctcccccacacccctgcccttgccagctgtgcccacctttgtatttagttttgtagtttcttggcttttataatcccctttttcttgccccctgggcttcggaggggggtgcttgtgcccccaatccccatgctcttgtgccttccccctctggccaggcctctgggctccgtgggggtgccctttctaggagggcagggttggacactgacggactgcaggcagggaggtagccacctggccctgccccctcctcatcccttcacccatccccaggactgctcgtccactatcatcactgtttttaatgcttttgtgttgattttttttaaagctgtcaactcattttcatctgttttttttttttgagaaaaaaaaaatgggaaaatgtacaaggcagccccttcccaggctttctgagcctggcctgacccagtctaagagggcctggggcaggatgtggccagccaggaaacataggatggcatttcttttatagactccttagtagttcaggggagtgggggagggtaggccagggctatttctgcccaccgaggagagaagagcgccaccctcctttttggaccctcatgagacaatgctgttcctagcagtggggtagtgactaccacccttctaccaaaaaaaaaaaaaaatcccttccttgtgggattcttgggcatctcctgcctccttcactctcacggtaattaatgtcttaattggctgttgcctggggaacaggagagctgctgcaggcagatgacctcatggggggtggagggaggtgaggtgcccaggtggctatttgccctgcagagctgggagttccccccaccaccaccaccctgttctctccttatctttggcatctttggttcaaggtggagatcttaagcgggtgtaaggccaggcggcctgctccttccctgggttctagtacaaagcacacacaccccccactcactcaatatggctcccactgtccccccaccccaccccccgccccgagtttggagctggggcctggaaagaggaagaggctgcttggggctgggccagcctgcagtgcactttgaccccagctccttggcagccacggctgctaacagactgccactcaactgtgcctcgcaggcactcccagggctgccatggaaggagctgggccttacaccacccacctccacactgcctcctggccagctgcccaccccggtgccaggtgggagagggagcaaaacagccagccccttccaggtggcagtcggaagggatttttgtttttgattctgttgccatttgtgtaaatactagtcttttttggaagaaataatgtaaagatgttttgtataaactctgaattattttcttgttgcttttttcttagaaaaaaaatgagaacc

SEQ ID NO: 20 - DNA, 4342 bp, Mus musculus
cccacgcgtccgcgcgcgctgagggacgcgcagggtgagcgcacctggcctcggcgaccgcgggagcggcgcggcgtgtccgccccggaggattgggggtcgctgcccgcggccggtcccaacgcgtcccggagtgcgcagaactgggagcggcttgggcatggccatggcccggtccaggagggactctgtgtggaagtactgttggggacttttgatggttttgtgcagaactgcgatctccagatcgatagttttagagcctatctactggaattcctcgaactccaaatttctacccggacaaggcctggtactatacccacagataggagacaaattggatattatttgccccaaagtggactctaaaactgttggccagtatgaatattataaagtttatatggttgataaagaccaagcagacagatgcacaattaagaaggagaataccccgctgctcaactgtgccagaccagaccaagatgtgaaattcaccatcaagtttcaagaattcagccctaacctctggggtctagaatttcagaagaacaaagattactacattatatctacatcaaatgggtctttggagggcctggataaccaggagggaggggtgtgccagacaagagccatgaagatcctcatgaaagttggacaagatgcaagttctgctggatcagccaggaatcacggtccaacaagacgtccagagctagaagctggtacaaatgggagaagttcaacaacaagtccctttgtgaagccaaatccaggttctagcaccgatggcaacagcgcggggcattccgggaacaatctcctgggttccgaagtggccttattcgcagggatcgcatcaggatgcatcatcttcatcgtcatcatcatcactttggtggtgctgctgctcaagtaccgcaggagacaccgcaaacactctccacagcacacgaccacgctgtctctcagcacactggccacgcccaagcgaggtggcaacaacaatggctcggagcccagtgacgttatcataccactaaggactgcagacagcgtcttctgcccgcactacgagaaggtcagcggggactatgggcacccggtgtacatcgtgcaggagatgcccccacagagtcctgccaacatttactacaaggtctgaggcctgagacctgcgcctcccaagggaactcgcaccttgttcttgggcacgcagggactgcctgagcctggctgtgggggcaggatgcctcctggaagagcctggatctggacagttttgtagtctgtagcttttccgaccctggggaccacagaccctccccggaagctggaagactgctaggagatccccacttggactgccgcggccccacgcggacctccaagccatgcacccagccactcaggcctctgcagagcccggggaggacacggtaggctatggatggcgcagcagcatcttaggagaaggttgcgcaccaggccggcccctgcctccacgtttcctgccgtgcacactggacttatcacttggacctcgggttcagtaaggttttcaaagatctctagtgtttagtcctcactcactcactcactcactcactccttctcttgccagggctctgcagcaaactccctagacccctcactccacgtactgcatcattacgggacactcaccacagagtcccagctccaccctttacaccaagatcaaattagatgggtattaggtacagaagaaccctgcttgcctggaggccgggtcagccgggaagcgcagatgtgtggaggagtgaggaggtgctggctgccacggcaggtcaaggctgcttgctgcccctggagcatagtagggatgcaggaaggaaatagataatgctttggtttttctgagaggacagagacaggtgggaggtgactgactggtgagtggtggggagcctttcactaccacacagctatgcagcagggaatcaaaagtccctctcctgcggggaacaaaggggccattgttgtgaaaggaccagctagagcacagagggagagggcaggcctccggtgaagtgctggggcagaactgcagaggtactggaaataaaaagcgcagcgcagagctgtgggagagtccgtctgctttgggagatgttttaagcagactcagctgctatattaccacgtttttattaaaaacacagggaaagcatttaggagaagagcagagagccaaatctgacctagaagttgaaaagccaaaggtcaaacaggctgtaagtccatcaccactgaggttattggagaattctcattaggaaaggcaggtcagattccccaggccccataagtgccccttccccctccctgattgagccttacacgttggtttttggtttatggccgtgctgtccgggctccaaggcagtacccgggctccatgtcaaagcaaagcacacatggcccacctcttagagtccttgagatggaagtaagttatgccgcggaaggaaaggcgaagataggacatatttataataggtgtatagaacacaagggatataaaatgaaagatttttactaatatatattttaagattacacacaatacacaccagaagacgtggagttcggtggtggtggtggtcgtggtggtggtgattaaagtgaccccagcgcttagtgctttaaaaagtgaaagattgggtagctactccccgaaacgtaccaatagcaagaaaagtatccataatgagagcaaatggcaaaaataacacggtcctgcgggaatctcgcagaaccgtagactaggaatgccagccccccaaattgatgtgaccctgccccgggttagacaatgataaaatgcgctggcctttattttctgtgttgggttttcccttgccttatgggctgaagtgttctctagaatttagcaggtcacactgaggggattccagtttaactgtgggtccctcctcctctcctaccccatccctgcccttccagagaataacaggaagccttcctttttttttttttttaagtgctatgcaaaagagacatctttaacagagtcctgttactatggtaacattttgctttctgaattgggaggaaataaaaattgtaatgacagcatttgaaggttctcagacctccagtgagtacctgcaaaaatgagttgtcacagagattattccctacttctcaaacctgaaaatgatgttggttcgatgtgtgtgtgtgtgtatgagtgggtgtgtggtacatgtgtgtacatatatgtataatatatatctccagtatatattatatatatctatatcatatttctgtggagggttgccatggcaatcaactgcagtacatatgtagttctttccatcaccctaacctctcctgcgcattcacacaagagtttcttgtaagccatcaaaagttaattctagggggagagggatgaggcggggagacatgggaaaccgtctgattttaatgaaatcaaatgtctgtgtcatcggttggctacgttttggttctatgctaaactgtgaagaatcggatgaattgatgaagagttgagttacctgcaacccattgagaagtgtcctgtgcgtctgttttgtgtctggtgcagaaaatgacaatctaccaactgtccccttatttggagttggttcagctttggaaagttactgtaaatgccttgcttgtattatcatccctagtcacctgacttcggagcttgcaccatcgtgttttaagtgaagacgctgtaaataggttcagatcttaccgtctatggattcgggtgttacagtagccttattcacctttttaataaaaatacacatgaaaacgagacagtaatggcttttcttacccagattgtgtacatagagcaatgttggttttttataaagtctaagcaagatgttttgtataaaatctgaattttgcaatgtatttagctacagcttttaacggcagtgtcatcccctttgcactgtaatgaggaaaaaaaaaaggtataaaaggttgccaaattgctgcatatttgtgccgtaattatgtaccatgaatatttatttaatttcgttgtccaatttgtaagtaacacagtattatgcttgagttataaatattttttctttgttttattttaatagcctgtcataggtttttttttaatctgctttagttccacatgacagttaagccccagaaatgagatccgagcagccacattccacgtctgtttcaaaatgaatttgttcttaaaaaaaataaaatatttttttcctatggaaaaaaaaaaaaaaagggcggccgc

SEQ ID NO: 21 - DNA, 3126 bp, Mus musculus
gaatcccggcgaggctggggctccagctcggtgtctccttcttcgctcccttgcccggcgcctcatgttgcccccgcccggccctcggctccccatgtcccccacgtagcggggcgcacaaatcccgaggtgcggcgcgtgggccgggggcgcgtaaggcgcctgcagacggcccctgcaagggttcttgtggggctgagcgctctggagccggggagcaagcatcgcagaaaagagggtccttgtgggagctgggggtgactagccacccaccagggtgactggggttccacagcgtggtagccacccctgggcggtgagagactttgggggagttggtgccccgcccccaggcccttggtagggtcatgggggccccccattttgggccagggggtgtgcaagtcggggccctgctgctgttaggttttgcggggctggtatctggactcagcctggagcctgtctactggaactcggcgaataagaggttccaggcagagggcggttacgtgctttatcctcagatcggggaccggctagatctactttgtccccgggctcggcctcctggcccccactcctctcctagttatgagttctacaaactgtacctggtagagggtgcccagggtcggcgttgtgaggcaccccctgccccaaaccttcttctcacatgtgaccggccagacctggacctccgcttcaccatcaagttccaggaatacagccctaacctctggggccacgagttccgatcccaccacgattactacataattgccacatcagacgggacccgggaaggccttgagagcttgcaaggaggcgtgtgcctaaccagaggcatgaaggtgcttctgcgagtgggacaaagtccccgaggaggagctgtaccccgaaaacctgtgtctgaaatgcccatggagagagacagaggggcagctcacagcgcggaacctgggagggacaccataccaggtgaccccagcagcaatgcaacctcccggggtgctgaaggccccctgccccctcccagcatgcccgcagtggctggggcagcaggggggatggcgctgctcttgctgggcgtggcaggggctgggggtgccatgtgttggcggagacggcgggccaagccttcggagagtcgccaccctggtcctggctcctttgggaggggagggtctctgggcctgggtggtggaggagggatggggcctcgggaagctgagcctggggagctaggaatagccctgcggggtggtgggactgcagacccccccttctgccctcactacgagaaggtgagcggtgactatgggcaccctgtgtacattgtgcaggatgggcccccccagagccctccgaacatctattacaaggtatgagggctcctcccacctggctctcctcaatctagccgtccatggggtgctcctccagtgtaattcatggtatgagggacacctgcgccatgactttggccccctttgccctaccagctctttggctcctcctggctatagccctcatctccaattttaggatcccttaagacttgactacaccttttcctgacctccccgactgaccctcatttgactgtgggcactcctctgtttctgcctcttaggggaggggggtcacaggctcagcctcccttaatcactacccaggtgtcctttctccctcacccacccagagtgggggtggggacagtctgtcttttggttggcacctctttctgcctctcaccatttttctcttctctctatttcttattctctctctctcctgtgtctaggtctgttctcttcctcctccctagcctcctccttacatctccttttaccctctcggcttcttgtccttcctgtgcctctcacgtgtctcctgggttggggcatcaaagcatctctcctctttgctcttggccctttccaacctttcataccaactgctcccctctgtccgccaaaaacgggggcttgcggggggtgggggggagattctggaactctacccccccagctccaggcatgggcacagggcctctttccctgcctttctgtgtacttactccagccatttggggtggttattgggccacaactactaccatgagaagaagtgtcctgttgtggccagtggctgataggaagacatgaagcagtgtggacatgatggactgtgtctgatgccgaatgggcatcagataggaagtgactcgttccagacaagaagtgaccaggcctggacagaaatggcctgggaagtgtccgcagcaggaactggaagtgccttcatccagaacaggaagtcgcacttctgaaataggaagtggtctggctggaattccaagtggcttagtctggggaggtggcctgggtgggggtgggggcatgggaagtggatggttcttaccctgtggagaaaggggcaggaggaactccctgctttaggaggaagctggaacttaccactgtgagaggacagatgtggactgagagttttcttagtgttcagtggcacttcccaaggaccccttcccttgtgctctgtgcagtttttaggacagctaagatgactgccacttgctgtggcaggcccaatttgtcttgttctccccttactgtaccccagtataatctctgttgatcaacagaactaccccaagaacccacatgttctccccacctaaccagacggctgtctggctgatgccttcctcccttcaagcccaacacagcgcccttgtcagtgaagaggtggtccatggactggtctagctgatggcagttattagcactggttgctgtttccatagtgacaagcctatacctcttcccgctctttagtgtgcagtgggctgctgcctcggtgttctcccgcctcagttttattagatcaaagctgcccttggccatcatgttggccacctcaatcaccagccaaaatggttgctttgtccaccagaggtcaagccatctctctggcgctgtagttcccagctccttctagggaacaggaagttgatattgccattggggaggtggcggggtatggccgtcacctcgatagttttattgtaaaagggaaatttgaacaacaacaaaaaaactg

(19) United States
(12) Patent Application Publication
(10) Pub. No.: US 2008/0031912 A1
(43) Pub. Date: Feb. 7, 2008

(54) METHOD FOR CONTROLLING CELL MIGRATION ON A SURFACE

(75) Inventors: Shu Chien, LaJolla, CA (US); Daniel Fero, LaJolla, CA (US)

Correspondence Address: KNOBBE MARTENS OLSON & BEAR LLP, 2040 MAIN STREET, FOURTEENTH FLOOR, IRVINE, CA, 92614, US

(73) Assignee: THE REGENTS OF THE UNIVERSITY OF CALIFORNIA, Oakland, CA (US)

(21) Appl. No.: 11/744,171

(22) Filed: May 3, 2007

Related U.S. Application Data
(60) Provisional application No. 60/798,811, filed on May 4, 2006.

(51) Int. Cl. A61K 38/02; A61F 2/00; A61P 43/00; C12N 5/06

(52) U.S. Cl. 424/422; 435/325; 514/2

(57) ABSTRACT

A method for directing or inhibiting cell migration on a two- or three-dimensional surface by contacting the surface with ephrin, peptide fragments derived from full-length ephrin, or synthetic peptide/small molecule agonists of Eph receptor tyrosine kinases. These surfaces include implantable, biocompatible devices which need to be completely or partially cell-free.

### CROSS-REFERENCE TO RELATED APPLICATIONS

This application claims priority under 35 U.S.C. 119(e) to U.S. Provisional Application No. 60/798,811, filed May 4, 2006, the entire contents of which are incorporated herein by reference.

### STATEMENT REGARDING FEDERALLY SPONSORED R&D

The present invention was supported by grant numbers HL 64382 and HL 080518 from the National Institutes of Health (NHLBI). The government may have certain rights in the invention.

### REFERENCE TO SEQUENCE LISTING

The present application is being filed along with a Sequence Listing in electronic format. The Sequence Listing is provided as a file entitled UCSD15-001A-SEQUENCELISTING.TXT, created May 3, 2007, which is 56.2 Kb in size. The information in the electronic format of the Sequence Listing is incorporated by reference in its entirety.

### BACKGROUND OF THE INVENTION

1. Field of the Invention

The present invention relates to a method for directing cellular migration on a bioactive surface/matrix via the use of ephrin proteins, fragments thereof or Eph receptor tyrosine kinase agonists.

2. Description of the Related Art

The Eph family of transmembrane receptor tyrosine kinases and their cognate ligands, ephrins, both constitute large families of cell surface signaling molecules that are prominently expressed by most cell types. The Eph tyrosine kinases have 14 members, including EphA receptors 1-8, 10 and EphB receptors 1-4, 6. The ephrins constitute an eight-member family of endogenous cellular proteins that mediate cell path-finding and directional migration via their interactions with Eph receptors. These members include ephrin A1-5 and ephrin B1-3. Ephrin ligand-Eph receptor interactions have been well characterized in terms of ligand/receptor binding promiscuity, and in resultant cell signaling changes.

Specifically, ephrins have been shown to play essential roles in shaping the nervous system and establishing vascular architecture during embryonic development (O'Leary et al., Curr. Opin. Neurobiol. 9:65-73, 1999; Adams et al., Trends Cardiovasc. Med. 10:183-188, 2000); Helbling et al., Development 127:269-278, 2000; Gale et al. Genes Dev. 13:1055-1066, 1999). Additional evidence has revealed a role in overall architectural remodeling at the cellular level, as demonstrated by the pronounced rounding and chemo-repulsive responses following Eph receptor activation (Murai et al., Nat. Neuro. 6:153-160, 2002; Zimmer et al. Nat. Cell Bio. 5:869-878, 2003; Miao et al. Nat. Cell Bio. 2:62-69, 2000). Ephrin receptors comprise the largest known family of receptor protein tyrosine kinases. They have been implicated in mediating developmental events, particularly in the nervous system. Receptors in the ephrin subfamily typically have a single kinase domain and an extracellular region containing a Cys-rich domain and two fibronectin type III repeats. Along with their ephrin ligands, they play important roles in neural development, angiogenesis, and vascular network assembly. Eph receptors have tyrosine-kinase activity, and, together with their ephrin ligands, mediate contact-dependent cell interactions that are implicated in the repulsion mechanisms that guide migrating cells and neuronal growth cones to specific destinations. Since Eph receptors and ephrins have complementary expression in many tissues during embryogenesis, bidirectional activation of Eph receptors may occur at interfaces of their expression domains, for example, at segment boundaries in the vertebrate hindbrain. Indeed, Eph receptors play key roles in development of the nervous system and angiogenesis. In the nervous system, they provide positional information by employing mechanisms that involve repulsion of migrating cells and growing axons (Frisen et al., 18(19) EMBO J. 5159-5165 (1999)). Also, an important function of Eph receptors and ephrins is to mediate cell-contact-dependent repulsion.

The current technology for using non-native implanted materials as cellular scaffolds employs coatings of engineered peptides or bioactive molecules upon which cells adhere (Fittkau et al., Biomaterials 26:167-174, 2005; Delong et al. Biomaterials 26:3227-3234, 2005). Similarly, implanted medical devices are manufactured with non-fouling synthetic surfaces to prevent bio-fouling and mitigate in vivo host-immune response. It is these surfaces, rather than the devices themselves, that cells sense and respond to.

Thus, there remains a need for compositions and methods which can direct cell migration on bioactive surfaces. The present invention addresses this need.

### SUMMARY OF THE INVENTION

The present invention provides a method for directing or inhibiting migration of cells on a surface by contacting the surface with ephrin, an ephrin peptide fragment, or an Eph receptor tyrosine kinase agonist, and contacting the surface with the cells, whereby migration of the cells is directed or inhibited. In one embodiment, the surface is two-dimensional. In another embodiment, the surface is three-dimensional. The ephrin, ephrin peptide fragment, or Eph receptor tyrosine kinase agonist may be incorporated within the three-dimensional matrix. In one embodiment, the surface is biocompatible. The ephrin, ephrin peptide fragment or Eph receptor tyrosine kinase agonist may be covalently or non-covalently bound to the surface. In another embodiment, the ephrin, ephrin peptide fragment or eph receptor tyrosine kinase agonist is conjugated to a ligand, and the surface is coated with the binding partner of the ligand. In one aspect of this embodiment, the ligand is biotin and the binding partner of the ligand is streptavidin. In yet another embodiment, the ephrin, ephrin peptide fragment or Eph receptor tyrosine kinase agonist is conjugated to an Fc antibody fragment, and the surface is coated with an antibody that binds the Fc fragment.

The present invention also provides a composition for directing or inhibiting cell migration, said composition comprising a surface and ephrin, an ephrin peptide fragment, or an Eph receptor tyrosine kinase agonist on said surface. In one embodiment, the surface is two-dimensional. In another embodiment, the surface is three-dimensional. The ephrin, ephrin peptide fragment or Eph receptor tyrosine kinase agonist may be incorporated within the three-dimensional matrix. In one embodiment, the surface is biocompatible. The ephrin, ephrin peptide fragment or Eph receptor tyrosine kinase agonist may be covalently or non-covalently bound to the surface. In another embodiment, the ephrin, ephrin peptide fragment or eph receptor tyrosine kinase agonist is conjugated to a ligand, and the surface is coated with the binding partner of the ligand. In one aspect of this embodiment, the ligand is biotin and the binding partner of the ligand is streptavidin. In yet another embodiment, the ephrin, ephrin peptide fragment or Eph receptor tyrosine kinase agonist is conjugated to an Fc antibody fragment, and the surface is coated with an antibody that binds the Fc fragment. In one embodiment, the surface is adapted to be implanted in the body.

### BRIEF DESCRIPTION OF THE DRAWINGS

FIG. 1 shows that NIH3T3 cells were able to spread efficiently on fibronectin (FN). In contrast, proper spreading was markedly inhibited on the ephrin-coated surface. Pictures were taken of the same field of view at 3, 6, and 8 hours post-plating.

FIG. 2 shows the migration inhibitory activity of Ephrin. The right halves of the surfaces of tissue culture dishes were coated with 1 μg/cm² ephrin A1 alone (top row), a mixture of 1 μg/cm² ephrin A1 and 1 μg/cm² FN (middle row), or 1 μg/cm² FN alone (bottom row), while the left half of the surface remained uncoated. The coating/no-coating interfaces are indicated by the vertical black lines. NIH 3T3 cells were then seeded on the uncoated portion (left half) and allowed to migrate for 7 days, while monitoring their location relative to the coating/no-coating interface.

FIG. 3 shows that ephrin A1-induced cellular de-adhesion and retraction is pik3R2-dependent. Wildtype and pik3R2^{-/-} MEFs were treated with 2 μg/mL of ephrin A1 for the indicated times and 4 monitored via time-lapse DIC microscopy. Wildtype MEFs undergo de-adhesion and retraction over the 30-minute time course, as indicated by arrows (upper panel). Conversely, pik3R2^{-/-} MEFs do not experience de-adhesion or overall retraction (lower panel). FIG. 3B shows the quantification of cell area over time for each cell type, based on pixel area, and normalized as percent of the original area recorded at time=0 minute. Each time point represents mean±SEM of cell area normalized to the original area. *p<0.05 compared to respective controls (0 time) for each cell line. # p<0.05 between wildtype and pik3R2^{-/-} cells at matched time points.

FIG. 4 shows that ephrin A1-induced actin cytoskeleton rearrangement is pik3R2-dependent. Wildtype and pik3R2^{-/-} MEFs were stimulated with 2 μg/mL ephrin A1 for indicated times, fixed with 2.5% paraformaldehyde, and stained with rhodamine-conjugated phalloidin. Cells were imaged with fluorescence microscopy to visualize the actin cytoskeleton structure. Wildtype MEFs exhibited marked cell retraction and actin cytoskeleton rearrangement following ephrin treatment, as indicated by arrows (upper panels). Conversely, pik3R2^{-/-} MEFs exhibited no retraction or actin rearrangement over the 30-minute time course (lower panels). Results are representative of 3 independent experiments.

FIG. 5 shows that ephrin A1-induced Rho activation is pik3R2-dependent. Wildtype and pik3R2^{-/-} MEFs were stimulated with 2 μg/mL ephrin A1 for the indicated times, and their cell lysates were subject to the RBD binding assay, SDS-PAGE, and immunoblotting for GTP-Rho and total Rho. Wildtype MEFs exhibited increased Rho activity (GTP-Rho) following ephrin treatment, whereas pik3R2^{-/-} MEFs did not. Bar graph represents mean±SD of GTP-Rho normalized to total Rho (N=3). *p<0.05 compared to control.

FIG. 6 shows that ephrin A1-induced MLC2 phosphorylation is pik3R2-dependent. Wildtype and pik3R2^{-/-} MEF cell lines were stimulated with 2 μg/mL ephrin A1 for the indicated times, and their cell lysates were subject to SDS-PAGE and immunoblotting for p-MLC2 and total MLC2. Wildtype MEFs exhibited increased phosphorylation of MLC2 following ephrin treatment, whereas pik3R2^{-/-} MEFs did not. Bar graph represents mean±SD of phosphorylated MLC2 (p-MLC2) normalized to total MLC2 (N=3). *p<0.05 compared to control.

FIG. 7 shows that ephrin A1-induced paxillin dephosphorylation is pik3R2-dependent. Wildtype MEFs were stimulated with 2 μg/mL Ephrin A1 for the indicated times, and their cell lysates were subject to SDS-PAGE and immunoblotting for p-paxillin and total paxillin. Wildtype MEFs exhibited marked de-phosphorylation of paxillin following ephrin treatment. Bar graph represents mean±SD of p-paxillin normalized to total paxillin (N=3). *p<0.05 compared to control.

### DETAILED DESCRIPTION OF THE PREFERRED EMBODIMENTS

The Eph receptor-ephrin interaction constitutes a well-conserved endogenous system aimed at defining cell-cell and tissue-specific borders in vivo, by controlling cell migration. It is this control over migration/tissue patterning that makes the Eph receptor-ephrin system an attractive candidate for functional use in bioactive surface engineering as a method to control cell migration and tissue patterning.

The present invention relates to manipulation of Eph receptor-ephrin interactions by using ephrin(s), peptide fragments derived from full-length ephrin protein sequences, or synthetic peptide/small molecule agonists of Eph receptor tyrosine kinases, as a bioactive component of a surface designed to remain completely or partially cell-free. These proteins/peptides/small molecules target Eph receptor-ephrin interactions and/or intracellular signaling specifically associated with cell adhesion, repulsion and migration. Mutant ephrins may also be used. These may be generated by mutagenizing the wild type protein, or by mutagenizing a nucleic acid encoding the protein by random mutagenesis or site-directed mutagenesis, both of which are well-known in the art. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304. PCR-based random mutagenesis kits are commercially available, such as the DIVERSIFY™ kit (Clontech).

The ephrin(s), peptide fragments derived from full-length ephrin protein sequences, or synthetic peptide/small molecule agonists of Eph receptor tyrosine kinases may be covalently or non-covalently attached to the surface/matrix. These surfaces/matrices may be two- or three-dimensional. The surfaces/matrices may also be biocompatible. In addition, the surfaces/matrices may be part of an implantable medical device. In one embodiment, the ephrin(s), peptide fragments derived from full-length ephrin protein sequences, or synthetic peptide/small molecule agonists of Eph receptor tyrosine kinases are patterned onto surfaces via injection into a mold. In another embodiment, the surface/matrix is immersed in, or sprayed with, a liquid solution of the ephrin(s), peptide fragments derived from full-length ephrin protein sequences, or synthetic peptide/small molecule agonists of Eph receptor tyrosine kinases.

The biocompatible matrix may also include a synthetic material including polyurethane, a segmented polyurethane-urea/heparin, a poly-L-lactic acid, cellulose ester, polyethylene glycol, polyvinyl acetate, dextran and gelatin; and/or a naturally-occurring material including collagen, elastin, laminin, fibronectin, vitronectin, heparin, fibrin, cellulose and amorphous carbon.

As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen of an organ, such as arteries, joints, bones, veins, ventricles or atrium of the heart. Any biocompatible, implantable medical device is suitable for use in the present invention. In one embodiment, a medical device is used which intimately (directly) contacts cells or tissues. Examples of such medical devices include without limitation, glucose sensors, pacemakers and pacemaker electrodes, stents, stent grafts, covered stents (such as those covered with polytetrafluoroethylene (PTFE), expanded polytetrafluoroethylene (ePTFE), or synthetic vascular grafts), artificial heart valves, artificial hearts and fixtures to connect the prosthetic organ to the vascular circulation, venous valves, abdominal aortic aneurysm (AAA) grafts, inferior venal caval filters, permanent drug infusion catheters, central venous catheters, urinary catheters, dialysis catheters, orthopedic implants, embolic coils, embolic materials used in vascular embolization (e.g., cross-linked PVA hydrogel), vascular sutures, vascular anastomosis fixtures, transmyocardial revascularization stents and/or other conduits, artificial joints, metal plates, rods, screws and the like.

Although humans are preferred, the coated medical devices may also be implanted into other mammals including, without limitation, humans, dogs, cats, horses, sheep, cows, rabbits, apes, rodents and the like.

The biocompatible medical devices may be made of one or more materials including, without limitation, stainless steel, polymers (e.g. polypropylene, polystyrene, polyester, polyethylene terephthalate, polytetrafluoroethylene), nickel-titanium, titanium, tantalum, gold, platinum-iridium, or Elgiloy and MP35N and other ferrous materials. In other embodiments, the biocompatible medical device is composed of polyurethane, cross-linked PVA hydrogel, biocompatible foams of hydrogels, or an inner layer of meshed polycarbonate urethane and an outer layer of meshed polyethylene terephthalate It will be apparent to those skilled in the art that ephrin(s), peptide fragments derived from full-length ephrin protein sequences, or synthetic peptide/small molecule agonists of Eph receptor tyrosine kinases may be applied to any biocompatible medical device. In some embodiments, medical devices can be used for end-to-end, end to side, side to end, side to side or intraluminal, and in anastomosis of vessels or for bypass of a diseased vessel segments, for example, as abdominal aortic aneurysm devices.

The ephrin(s), peptide fragments derived from full-length ephrin protein sequences, or synthetic peptide/small molecule agonists of Eph receptor tyrosine kinases are applied to one or more surfaces of these medical devices, or portions thereof, as described herein. The medical device may be coated with these compounds prior to a surgical procedure to implant the device, or may be coated during the surgical procedure.

As used herein, the term “ephrin proteins” refers to the full-length proteins, as determined by the nucleotide cDNA sequence listed in GenBank or any other publicly available database. Exemplary Ephrin sequences include, but are not limited to, those listed under GenBank Accession Numbers NM 004428, NM 182685, NM 001405, NM 004952, NM 005227, NM 182689, NM 182690, NM 001962, NM 004429, NM 004093, NM 001406, for human sequences, and NM 010107, NM 007909, XM 910035, XM 892839, NM 007910, NM 207654, NM 010109, NM 010110, NM 010111 and NM 007911 for mouse sequences, the entire contents of which are incorporated herein by reference. These sequences are shown in Appendix A. Although exemplary ephrin nucleotide sequences are provided herein in Appendix A, it will be appreciated that other nucleic acids which encode polypeptides which are at least 50%, 55%, 60%, 65%, 70%, 75%, 80%, 85%, 90%, 95% or 99% identical to these sequences, and which encode ephrins which retain the ability to direct or inhibit cell migration, may also be used in the compositions and methods described herein. The term “ephrin” also encompasses any modified, full-length ephrin protein which may alter protein function, but not the ability of the protein to function as an Eph receptor agonist. The term “peptide fragment” refers to any peptide fragment derived from the full-length wild type ephrin protein, or a mutant ephrin protein. These fragments may also be modified as discussed above, while retaining their ability to act as agonists towards Eph receptors. The term “Eph receptor tyrosine kinase agonist” refers to any synthetic protein/peptide sequence, or small molecule, that can act as an Eph receptor agonist. These include, but are not limited to, antibodies, fusion proteins of multiple ephrin ligand binding domains or small molecule structures. The identification of such compounds can be performed using methods well known in the art, including competitive binding assays, Eph activity assays and phosphorylation assays.

An ephrin-coated surface/device will interact with contacting cells and functionally prevent those cells from migrating into ephrin-coated regions via interactions between the ephrin coating, and the cell's own endogenous Eph receptors. This prevents unwanted cell migration by exploiting a cell's own anti-migratory signaling mechanism, thus providing a way to functionally inhibit cell migration onto designated surfaces. Because the growth and migration of cells onto surfaces of medical devices may cause problems by negatively impacting the function of the medical device, the ephrin(s), peptide fragments derived from full-length ephrin protein sequences, or synthetic peptide/small molecule agonists of Eph receptor tyrosine kinases will prevent such unwanted cell growth and migration. In other applications, it may be desirable to allow cell growth on certain parts/components of a medical device, but to keep other parts/components cell-free. In this case, ephrin(s), peptide fragments derived from full-length ephrin protein sequences, or synthetic peptide/small molecule agonists of Eph receptor tyrosine kinases can be applied to the areas desired to be cell free. These compounds will thus inhibit cell growth and migration on these particular areas, but growth/migration will still occur and be directed to different areas of the implantable device.

A single recombinant ephrin protein subtype, combinations of multiple (two or more) ephrin protein subtypes, recombinant peptide fragments of one or more ephrin protein subtypes, synthetic peptide/small molecule, Eph receptor tyrosine kinase agonists or any combination of the above can be attached to a surface via covalent or non-covalent bonding, or included as a component of a 3-dimensional bioactive polymer matrix. The ephrin/ephrin fragment/agonist interacts with endogenous cellular Eph receptors to selectively activate a chemorepulsive response in cells upon contact, thereby inhibiting cellular migration into regions containing the ephrin/ephrin fragment/agonist. This leads to selective prevention of cell migration onto ephrin/ephrin fragment/agonist-containing surfaces of a two-dimensional bioactive surface or a three-dimensional bioactive matrix. These surfaces may be made out of any biocompatible material including metals (e.g., steel, titanium), nylon, polycarbonate, ceramic, glass, and the like, and may be performed in vitro or in vivo.

Ephrin proteins/fragments/Eph receptor tyrosine kinase agonists can be adsorbed to, coated onto, bonded to or incorporated into a two- or three-dimensional surface/matrix through a variety of means, resulting in the ability of these molecules to freely interact with endogenous cellular Eph receptors. Both the placement and/or orientation on the surface/matrix can be controlled. The method of attachment includes, but is not limited to, passive adsorption, covalent linkage, noncovalent linkage and antibody conjugation. The amount of ephrin(s), peptide fragments derived from full-length ephrin protein sequences, or synthetic peptide/small molecule agonists of Eph receptor tyrosine kinases attached to a surface/matrix will vary, depending on the particular compound and surface/matrix used, and can be empirically determined by one of ordinary skill in the art. In some embodiments, the amount of ephrin(s), peptide fragments derived from full-length ephrin protein sequences, or synthetic peptide/small molecule agonists of Eph receptor tyrosine kinases present on the surface matrix is between about 0.01 and 100 μg/cm², between about 0.1 μg/cm² and 50 μg/cm² or between about 1 μg/cm² and 25 μg/cm².

In one embodiment, recombinant ephrin protein/peptide fragments/related agonists are patterned onto surfaces via injection into a mold (e.g. a polydimethylsiloxane (PDMS) mold). Briefly, a silicone wafer is spin-coated with photoresist (e.g., SU-8, Microchemical Co., MA), and a mask aligner is used to expose the wafer to ultraviolet light through the mask with a pre-printed pattern. The unexposed photoresist is washed away during the development process, leaving behind a microfabricated template for the PDMS mold. The PDMS mold is prepared according to the manufacturer's instructions (Sylgard 184, Dow, Corning, MI), degassed under vacuum, cast on the patterned wafer and baked for 2 hours at 70° C. The mold is subsequently sealed on the desired surface and the resultant micro-channels between the PDMS mold and the surface are used for microfluidic patterning of ephrin. The ephrin/peptide fragment/related agonist solution (1 μg/ml) is introduced and incubated in the microchannels for 2 hours. The non-coated areas are subsequently passivated by incubation with 1% F108 Pluronic solution (BASF, triblock polyethylene oxide-polypropylene polymer) in water overnight.

The recombinant ephrin protein/peptide fragments/related agonists may comprise a conjugated peptide tag. In one embodiment, the tag is streptavidin, which is specific for conjugation by biotin. The tag can also be a receptor which binds to its cognate ligand, a ligand which binds to its cognate receptor, or any molecule which has a counterpart to which it binds. Following biotinylation of the recombinant ephrin protein/peptide fragments/related agonist, it can be adsorbed to or bound to a streptavidin-coated surface, streptavidin-containing polymer, or streptavidin-linked molecule. This allows increased binding strength of the biotin-conjugated protein to the streptavidin molecule (K_{d} approx. 10⁻¹⁵ M), and increased control over protein orientation with respect to the streptavidin binding surface/biotin conjugated peptide tag.

In another embodiment, the recombinant ephrin protein/peptide fragments/related agonists comprises an Fc antibody conjugate. This Fc tag is then used to adhere the recombinant ephrin protein/peptide fragments/related agonist to an antibody-coated surface. This allows for greater binding affinity between the ephrin/similar peptide and the antibody coated surface, and allows for increased control over a protein orientation with respect to the surface.

Similar methods may also be used as a way to incorporate recombinant ephrin protein/peptide fragments/related agonists into a bioactive surface/matrix. This may be done, for example, through genetic fusion of various protein sequence tags or molecular conjugation of reactive groups to the recombinant ephrin protein/peptide fragments/related agonist.

The present methods are useful for the design of bioactive surfaces intended for implantation into a patient, whereupon the surfaces will interact with local tissues to direct and/or prevent cellular migration onto the surfaces. The methods described herein are also suitable for the design of implantable tissue-engineered matrices aimed at controlling the direction/orientation of cell migration and/or growth.

The present invention can be used alone, or in combination with additional pro-migratory tissue engineering technology to specifically designate paths and patterns for cell growth and/or migration, whereupon the ephrin/ephrin fragment proteins would confer directional cues through chemorepulsive signaling effects on local cells, i.e. implantable tissue engineering matrices to direct paths for axon regeneration.

The present methods also can be used to provide optimum geometric patterns of cell hybrids, e.g. in tissue-engineered grafts, by controlling the growth of component cells; to direct angiogenesis so that endothelial cells migrate away from regions where angiogenesis is undesirable, and be directed toward areas that require angiogenesis; biopharmaceutical applications in which cell migration and/or localization are directly controlled for the purpose of the application (e.g., segregation of cell populations in vitro such as cellular co-culture where two or more cell populations are to share fluid media, but are not in contact with one another; and for cell separation by use of the chemorepulsive properties of the ephrin-Eph receptors in conjunction with chemoattractive molecular systems.

In one embodiment, ephrin, an ephrin peptide fragment, or an Eph receptor tyrosine kinase agonist is used to divide tissue culture flasks or dishes into two or more sections, each section containing a different cell type (or the same cell type may be present in two or more of the sections). For example, if the objective is to grow two different cell types on a single plate, or in a single flask, in the same media and not have the two cell types intermingle, then a “line” of ephrin, an ephrin peptide fragment, or an Eph receptor tyrosine kinase agonist is used divide the plate into two sections. One cell type is then applied to one side of the plate, and the other cell type is applied to the other side of the plate. Because the ephrin, an ephrin peptide fragment, or an Eph receptor tyrosine kinase agonist applied to the plate inhibits cell migration and adhesion, the two cell types do not expand beyond the region coated with ephrin, an ephrin peptide fragment, or an Eph receptor tyrosine kinase agonist and will thus not intermingle. Cell culture plates may also be divided into a plurality of sections using a plurality of ephrin, an ephrin peptide fragment, or an Eph receptor tyrosine kinase agonist “lines” in order to test the effects of different compounds of interest on a single cell type or multiple cell types (e.g., cytotoxicity, cell growth, cell morphology, and the like).

Cell culture dishes or flasks may also be “patterned” with ephrin, an ephrin peptide fragment, or an Eph receptor tyrosine kinase agonist in conjunction with patterning of chemoattractive molecules to promote cell separation. Cells that have ephrin receptors will be inhibited by the regions of ephrin, an ephrin peptide fragment, or an Eph receptor tyrosine kinase agonist, and will thus be directed away from these molecules, and toward the chemoattractive molecules, resulting in separation of a plurality of cell types.

Studies were performed in order to establish the functional effects of an ephrin A1-coated surface on cell migration and spreading as described below. To define the biochemical signaling that regulates this ephrin-induced inhibition of cell adhesion/migration, biochemical assays aimed at identifying critical signaling elements that regulate the observed behavior were conducted. These studies are described below.

Materials and Methods

Cell Culture. NIH 3T3 cells were cultured in DMEM supplemented with 10% calf serum, 1% sodium pyruvate, 1% L-glutamine, 1% penicillin/streptomycin, and maintained in a humidified 5% CO₂/95% air incubator at 37° C. Wildtype and pik3R2^{-/-} MEF cell lines were maintained in DMEM supplemented with 15% FBS and 1% penicillin/streptomycin, and maintained in a humidified 5% CO₂, 95% air incubator at 37° C.

Reagents. A recombinant mouse Ephrin-A1/Fc chimera was purchased from R&D systems (Minneapolis, Minn.). It is comprised of the extracellular domain of mouse ephrin A1, (AA residues Met1-Ser182) fused to the carboxy-terminal 6× histidine-tagged Fc region of human IgG, via a polypeptide linker. The Ephrin-A1/Fc chimera was reconstituted in PBS to a concentration of 200 μg/mL and stored at −20° C.

Imaging. NIH 3T3 cells were maintained in complete media (described above) for all experiments. Imaging was done on a Nikon Diaphot 300 inverted microscope with a Hamamatsu Orca ER digital camera controlled by IP lab software (Scanalytics). Images were captured under 10× and 20× phase-contrast microscopy. The cells were under temperature and gas control throughout the duration of the experiments (5% CO₂, 95% air, 37° C.). MEF cells were maintained in complete media for all experiments. Imaging was done on a Nikon Diaphot 300 inverted microscope with a Hamamatsu Orca ER digital camera controlled by IP lab, or Metamorph imaging software. Images were captured under 20× and 40× differential interference microscopy (DIC), or fluorescence microscopy. The cells were under temperature and gas control throughout the duration of live cell imaging experiments (5% CO₂, 95% air, 37° C.).

RBD Binding Assay. MEF cells were treated with ephrin A1 for various times, lysed, and incubated with GST-RBD (rhotekin binding domain for Rho) beads at 4° C. for 1 hour. The beads were then centrifuged for collection, washed, and subject to SDS-PAGE and immunoblotting.

SDS-PAGE and Immunoblotting. Proteins were separated based on their relative size using sodium dodecyl sulfate-polyacrylamide gel electrophoresis (SDS-PAGE). The lysates were reduced by the addition of a loading buffer and boiled for 10 minutes. The denatured proteins were loaded into 8, 10, or 12% cross-linked gels and separated by a voltage gradient. The separated proteins were then transferred to a nitrocellulose membrane (Bio-Rad, CA), and the membrane was blocked with 5% bovine serum albumin (BSA) in TBSt (TBS with 0.1% Tween −20) for 2 hours. The membranes were then be incubated with the specific primary and secondary antibodies to detect the proteins of interest.

Ephrin Inhibits Cell Spreading

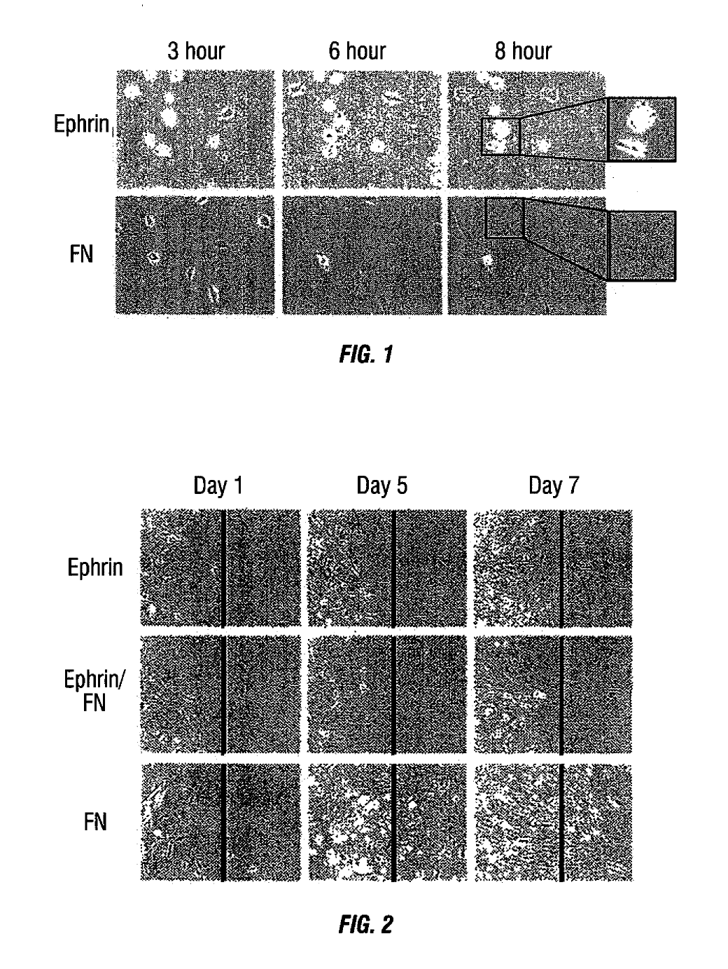

NIH 3T3 cells were seeded on surfaces of tissue culture dishes that were coated with 1 μg/cm2 of either ephrin A1 or fibronectin (FN), and cell spreading was observed over an 8-hour time period via time-lapse microscopy. As shown in FIG. 1, the cells were able to spread efficiently on the FN; in contrast, proper spreading was markedly inhibited on the ephrin-coated surface. Pictures were taken of the same field of view at 3, 6, and 8 hours post-plating. Ephrin A1 inhibited cell spreading throughout the 8-hour duration, whereas cells plated on FN were well spread by 3 hours. Insets from 8-hour frames are enlarged to show the extent of cellular spreading of representative cells.

Ephrin Inhibits Cell Migration

In FIG. 2, the right halves of the surfaces of tissue culture dishes were coated with 1 μg/cm² ephrin A1 alone (top row), a mixture of 1 μg/cm² ephrin A1 and 1 μg/cm² FN (middle row), or 1 μg/cm² FN alone (bottom row), while the left half of the surface remained uncoated. The coating/no-coating interfaces are indicated by the vertical black lines. NIH 3T3 cells were then seeded on the uncoated portion (left half) and allowed to migrate for 7 days, while monitoring their location relative to the coating/no-coating interface. For all time points, cell migration was inhibited by coating with ephrin A1 or a mixture of ephrin A1 and FN together. FN alone, however, allowed for robust migration onto the coated surface. The different cell densities observed between panels is due to random field selection.

The results demonstrate that ephrin A1, when used as a surface coating, acts as a potent inhibitor of both spreading and migration of NIH 3T3 cells, even in the presence of the pro-migratory extracellular matrix protein fibronectin (FN). This inhibition lasted for several days. These findings indicate that ephrin A1 is a suitable candidate molecule for precise inhibition of cell migration when used as a bioactive component of an engineered surface. This method exploits a cell's own endogenous mechanism of migratory inhibition as a way to functionally modulate cell behavior on an engineered surface. This offers precise control, and a higher degree of reproducibility when designing surfaces/matrices that require precise patterning and control of cellular growth and migratory pathways. It also increases biocompatibility, when dealing with implanted devices, where biofouling and aberrant cell migration have been problematic in the past. The utilization of this intrinsic cell-ligand contact signaling system to inhibit migration can be applied to novel applications in tissue and device engineering, and the incorporation of the Eph receptor-ephrin system into current design paradigms improves control over cell migration on an engineered surface.

Ephrin Induces Cellular De-Adhesion and Retraction in a Phosphatidylinositol-3 Kinase Beta (PI3Kβ) Dependent Manner

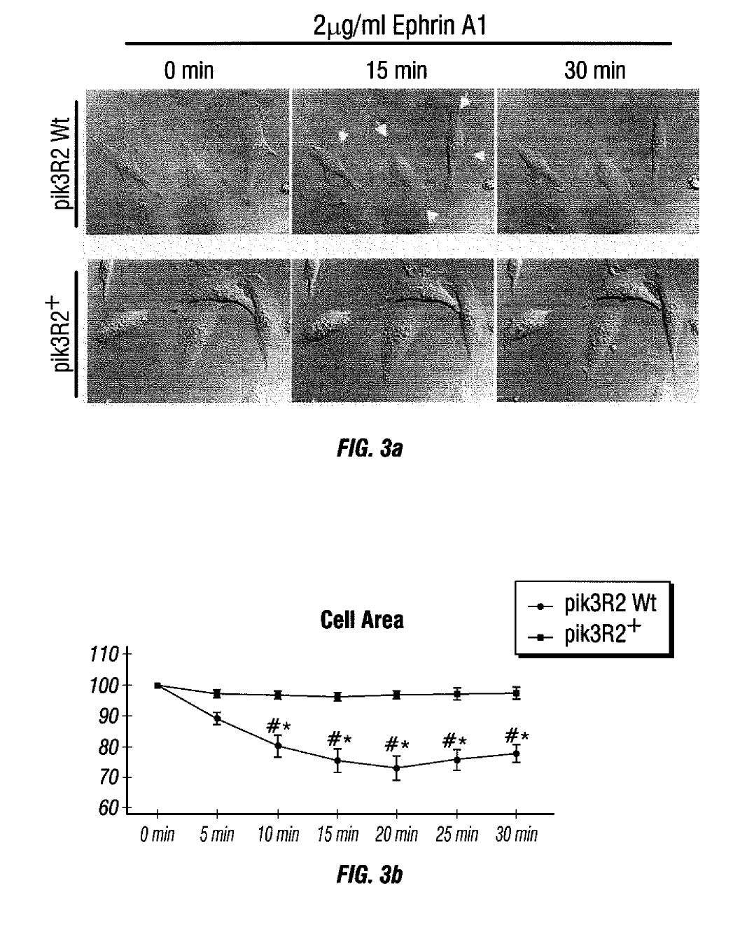

The PI3K family of lipid kinases are known to regulate both cell adhesion to and locomotion on a surface. Wildtype mouse embryonic fibroblasts (MEFs) and MEFs devoid of PI3Kβ enzymatic activity, achieved through genetic deletion of the pik3R2 gene (pik3R2^{-/-}), were treated with 2 μg/mL of ephrin A1 for 30 minutes. Cellular de-adhesion and overall retraction was monitored using time-lapse DIC microscopy. FIG. 3A shows that wildtype MEFs experience cellular de-adhesion and cell retraction over the 30-minute time course, indicated by arrows (upper panels). Conversely, pik3R2^{-/-} MEFs do not experience de-adhesion or overall cellular retraction (lower panels), indicating that the cell de-adhesion/rounding response to ephrin A1 is mediated through PI3K enzymatic activity. FIG. 3B shows the quantification of cell area over time, based on pixel area, and normalized as percent of the original area recorded at time=0 minute.

Ephrin Induces Actin Cytoskeleton Rearrangement in a PIK3β Dependent Manner

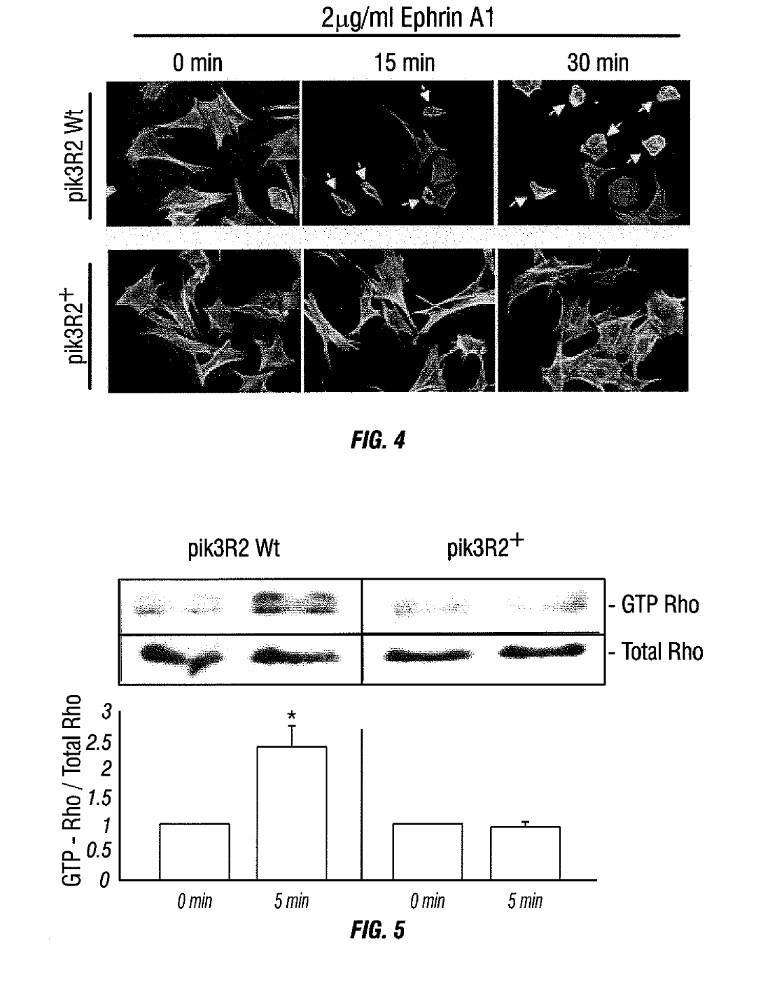

The actin cytoskeleton is known to regulate cell architecture and locomotion through changes in its structure following P13K enzymatic activity. To determine the role of ephrin A1 in inducing PI3K-mediated actin cytoskeletal changes, wildtype and pik3R2^{-/-} MEFs were stimulated with 2 μg/mL ephrin A1 for indicated times, fixed with 2.5% paraformaldehyde, stained with rhodamine-conjugated phalloidin, and imaged with fluorescence microscopy to visualize the actin cytoskeleton structure. Wildtype MEFs exhibited marked actin cytoskeleton rearrangement and cell retraction following ephrin treatment, as indicated by arrows (upper panel in FIG. 4). Conversely, pik3R2^{-/-} MEFs exhibited no retraction or actin rearrangement over the 30-minute time course (lower panel in FIG. 4), indicating that ephrin A1 induced changes to actin-based cell morphology and locomotion are mediated through PI3Kβ.

Ephrin A1 Induces Rho Activity in a PIK3β Dependent Manner

The small GTPase Rho regulates actin rearrangement and cellular contractility. To determine whether PI3Kβ mediates Rho activity to result in changes in actin structure and contractility following ephrin A1 stimulation, wildtype and pik3R2_{-/-} MEFs were treated with 2 μg/mL ephrin A1 for indicated times, and their cell lysates were subject to the RBD binding assay, SDS-PAGE, and immunoblotting for GTP-Rho and total Rho. Wildtype MEFs exhibited increased Rho activity (GTP-Rho) following ephrin treatment, whereas pik3R2^{-/-} MEFs did not (FIG. 5). These results indicate that ephrin A1 induced Rho activation is mediated through PI3Kβ.

Ephrin A1 Induces MLC2 Phosphorylation in a PIK3β Dependent Manner

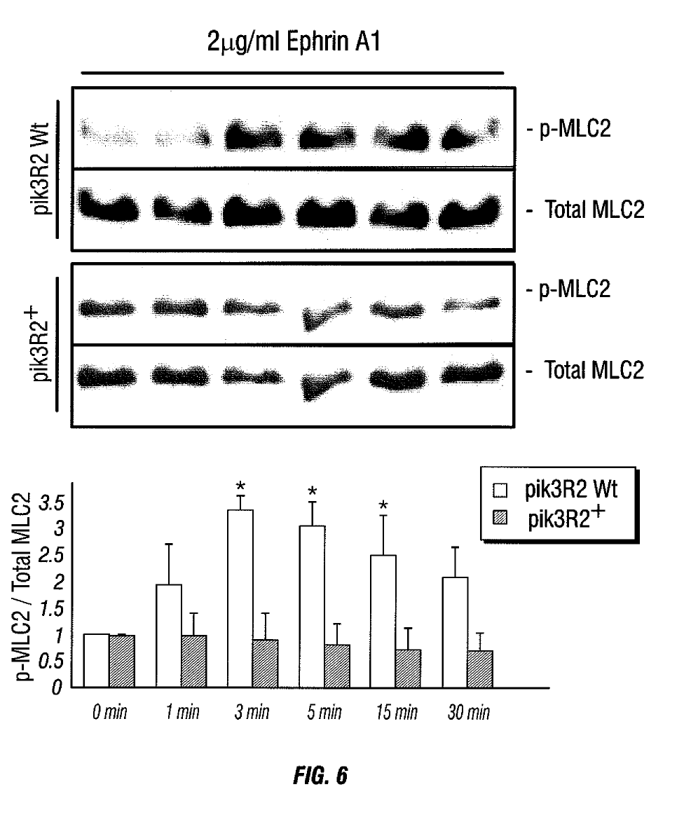

Myosin light chain 2 (MLC2) is a non-muscle cell contractile protein that mediates cell contractility and locomotion via its phosphorylation of Threonine 18 and Serine 19. To determine whether MLC2 is phosphorylated in a PI3Kβ-dependent manner following ephrin A1 treatment, wildtype and pik3R2^{-/-} MEFs were stimulated with 2 μg/mL ephrin A1 for indicated times, and their cell lysates were subject to SDS-PAGE and immunoblotting for p-MLC2 and total MLC2. Wildtype MEFs exhibited increased phosphorylation of MLC2 on Threonine 18 and Serine 19 following ephrin treatment, whereas pik3R2^{-/-} MEFs did not (FIG. 6). These results indicate that MLC2 phosphorylation, and thus increased cellular contractility, is mediated by PI3Kβ activity following ephrin A1 treatment.

Ephrin A1 Induces Paxillin Dephosphorylation in a PIK3β-Dependent Manner

Paxillin is a focal adhesion protein that provides the structural basis of cell attachment and adhesion to a surface when it is phosphorylated on Tyrosine 118. To determine the effect of ephrin A1 treatment on the phosphorylation level of paxillin, and thus cell attachment to its substrate, wildtype MEFs were stimulated with 2 μg/mL ephrin A1 for indicated times, and their cell lysates were subject to SDS-PAGE and immunoblotting for p-Paxillin (Tyrosine 118) and total Paxillin. Wildtype MEFs exhibited marked de-phosphorylation of Paxillin following ephrin treatment, indicating focal adhesion disassembly and cell de-adhesion from the surface.

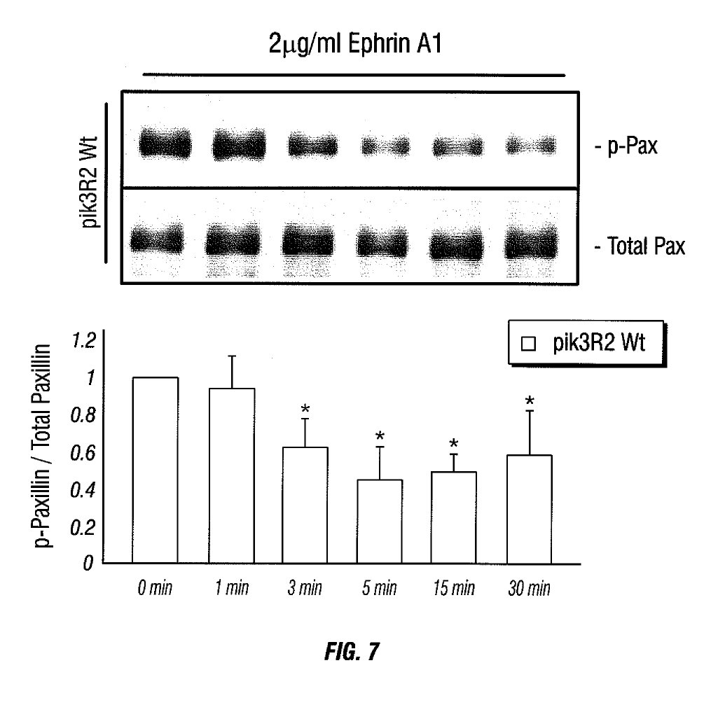

The results show that ephrin-Eph receptor signaling mediates both the process of cellular adhesion to its substrate (FIGS. 3 and 7) and overall cell morphology/migration FIGS. 3-6). These results define the biologic pathways by which an ephrin-coated surface is able to elicit anti-adhesion/migration effects upon contacting cells. By functionally mimicking cell-cell interactions, the ephrin coating is able to induce these biochemical changes that regulate adhesion and migration, and as such, functionally control cell behavior based on these predictable elements of molecular signaling.

These results demonstrate that the Eph receptor-ephrin signaling paradigm is a critical regulator of the molecular mechanisms that control cell migration and attachment. These mechanisms include PI3Kβ-mediated actin cytoskeleton rearrangement, Rho GTPase activation, and MLC2 phosphorylation, which regulate cell morphology and motility dynamics, and paxillin phosphorylation status, which regulates cellular attachment to a substrate through the formation and maintenance of focal adhesion complexes. These findings elucidate the biological effects induced by using ephrin as a bioactive component of an engineered surface. Functionally, this method is able to regulate cell behavior at the molecular level to inhibit attachment and migration onto coated surfaces in a way that mimics intrinsic in vivo cell-cell based inhibition of aberrant cell migration and attachment. This technology will allow for improved biocompatibility and precise control over cell migration on an engineered surface via the incorporation of ephrin proteins, or similar peptides or small molecules, to functionally regulate cell attachment and migration dynamics.

What is claimed is:

1. A method for directing or inhibiting migration of cells on a surface, comprising contacting said surface with ephrin, an ephrin peptide fragment, or an Eph receptor tyrosine kinase agonist; and contacting said surface with said cells, whereby migration of said cells is directed or inhibited.

2. The method of claim 1, wherein said surface is a two dimensional surface.

3. The method of claim 1, wherein said surface is a three dimensional matrix.

4. The method of claim 3, wherein said ephrin, ephrin peptide fragment, or Eph receptor tyrosine kinase agonist is incorporated within said matrix.

5. The method of claim 1, wherein said surface is biocompatible.

6. The method of claim 1, wherein said ephrin, ephrin peptide fragment, or Eph receptor tyrosine kinase agonist is covalently bound to said surface.

7. The method of claim 1, wherein said ephrin, ephrin peptide fragment, or Eph receptor tyrosine kinase agonist is non-covalently bound to said surface.

8. The method of claim 1, wherein said ephrin, ephrin peptide fragment, or Eph receptor tyrosine kinase agonist are conjugated to a ligand, and said surface is coated with the binding partner of said ligand.

9. The method of claim 8, wherein said ligand is biotin and said binding partner of said ligand is streptavidin.

10. The method of claim 1, wherein said ephrin, ephrin peptide fragment, or Eph receptor tyrosine kinase agonist is conjugated to an Fc antibody fragment, and said surface is coated with an antibody that binds said Fc fragment.

11. A composition for directing or inhibiting cell migration, said composition comprising a surface and ephrin, an ephrin peptide fragment, or an Eph receptor tyrosine kinase agonist on said surface.

12. The composition of claim 11, wherein said surface is two dimensional.

13. The composition of claim 11, wherein said surface is three dimensional.

14. The composition of claim 11, wherein said ephrin, ephrin peptide fragment, or Eph receptor tyrosine kinase agonist is covalently bonded to said surface.

15. The composition of claim 11, wherein said ephrin, ephrin peptide fragment, or Eph receptor tyrosine kinase agonist is non-covalently bonded to said surface.

16. The composition of claim 11, wherein said surface is biocompatible.

17. The composition of claim 13, wherein said ephrin, ephrin peptide fragment, or Eph receptor tyrosine kinase agonist is incorporated into said surface.

18. The composition of claim 11, wherein said ephrin, ephrin peptide fragment, or Eph receptor tyrosine kinase agonist are conjugated to a ligand, and said surface is coated with the binding partner of said ligand.

19. The composition of claim 18, wherein said ligand is biotin and said binding partner of said ligand is streptavidin.

20. The composition of claim 11, wherein said ephrin, ephrin peptide fragment, or Eph receptor tyrosine kinase agonist is conjugated to an Fc antibody fragment, and said surface is coated with an antibody that binds said Fc fragment.

21. The composition of claim 11, wherein said surface is adapted to be implanted in the body.
